SEQUENCE LISTING (5 sequences)

SEQ ID NO: 1 - DNA, 4445 bp, Homo sapiens
cgctccccgctcccgtggctgccgccgccccggggaagaagagacaggggtggggtttgggggaagcgagagaggaggggagagaccctggccaggctggagcctggattcgaggggaggagggacgggaggaggagaaaggtggaggagaagggaggggggagcggggaggagcggccgggcctggggccttgaggcccggggagagccggggagccgggcccgcgcgccgagatgttgctgctgctgttactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtgagggtagggtgagggaggacaggccagtagggggagggaaagggagaggggaagggcaggggactcaggaagcagggggtccccatccccagctgggaagaacatgctatccaatctcatctcttgtaaatacatgtccccctgtgagttctgggctgatttgggtctctcatacctctgggaaacagacctttttctctcttactgcttcatgtaattttgtatcacctcttcacaatttagttcgtacctggcttgaagctgctcactgctcacacgctgcctcctcagcagcctcactgcatctttctcttcccatgcaacaccctcttctagttaccacggcaacccctgcagctcctctgcctttgtgctctgttcctgtccagcaggggtctcccaacaagtgctctttccaccccaaaggggcctctccttttctccactgtcataatctctttccatcttacttgcccttctatactttctcacatgtggctccccctgaattttgcttcctttgggagctcattcttttcgccaaggctcacatgctccttgcctctgctctgtgcactcacgctcagcacacatgcatcctcccctctcctgcgtgtgcccactgaacatgctcatgtgtacacacgcttttcccgtatgctttcttcatgttcagtcacatgtgctctcgggtgccctgcattcacagctacgtgtgcccctctcatggtcatgggtctgcccttgagcgtgtttgggtaggcatgtgcaatttgtctagcatgctgagtcatgtctttcctatttgcacacgtccatgtttatccatgtactttccctgtgtaccctccatgtaccttgtgtactttcttcccttaaatcatggtattcttctgacagagccatatgtaccctaccctgcacattgttatgcacttttccccaattcatgtttggtggggccatccacaccctctccttgtcacagaatctccatttctgctcagattccccccatctccattgcattcatgtactaccctcagtctacactcacaatcatcttctcccaagactgctcccttttgttttgtgtttttttgaggggaattaaggaaaaataagtgggggcaggtttggagagctgcttccagtggatagttgatgagaatcctgaccaaaggaaggcacccttgactgttgggatagacagatggacctatggggtgggaggtggtgtccctttcacactgtggtgtctcttggggaaggatctccccgaatctcaataaaccagtgaacagtgtgactcggcaaaaaaaaaaa

SEQ ID NO: 2 - DNA, 4039 bp, Homo sapiens
agagccgcgggggccgtaggaagccaaccttccctgcttctccggggccctcgccccctcctccccacaaaatcagggatggaggcgcctccccggcaccctcttagcagccctccccaggaaaagtgtcccccctgagctcctaacgctccccaacagctacccctgccccccacgccatggggcccggggccccttttgcccgggtggggtggccactgccgcttctggttgtgatggcggcaggggtggctccggtgtgggcctcccactccccccatctcccgcggcctcactcgcgggtccccccgcacccctcctcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtgagggtagggtgagggaggacaggccagtagggggagggaaagggagaggggaagggcaggggactcaggaagcagggggtccccatccccagctgggaagaacatgctatccaatctcatctcttgtaaatacatgtccccctgtgagttctgggctgatttgggtctctcatacctctgggaaacagacctttttctctcttactgcttcatgtaattttgtatcacctcttcacaatttagttcgtacctggcttgaagctgctcactgctcacacgctgcctcctcagcagcctcactgcatctttctcttcccatgcaacaccctcttctagttaccacggcaacccctgcagctcctctgcctttgtgctctgttcctgtccagcaggggtctcccaacaagtgctctttccaccccaaaggggcctctccttttctccactgtcataatctctttccatcttacttgcccttctatactttctcacatgtggctccccctgaattttgcttcctttgggagctcattcttttcgccaaggctcacatgctccttgcctctgctctgtgcactcacgctcagcacacatgcatcctcccctctcctgcgtgtgcccactgaacatgctcatgtgtacacacgcttttcccgtatgctttcttcatgttcagtcacatgtgctctcgggtgccctgcattcacagctacgtgtgcccctctcatggtcatgggtctgcccttgagcgtgtttgggtaggcatgtgcaatttgtctagcatgctgagtcatgtctttcctatttgcacacgtccatgtttatccatgtactttccctgtgtaccctccatgtaccttgtgtactttcttcccttaaatcatggtattcttctgacagagccatatgtaccctaccctgcacattgttatgcacttttccccaattcatgtttggtggggccatccacaccctctccttgtcacagaatctccatttctgctcagattccccccatctccattgcattcatgtactaccctcagtctacactcacaatcatcttctcccaagactgctcccttttgttttgtgtttttttgaggggaattaaggaaaaataagtgggggcaggtttggagagctgcttccagtggatagttgatgagaatcctgaccaaaggaaggcacccttgactgttgggatagacagatggacctatggggtgggaggtggtgtccctttcacactgtggtgtctcttggggaaggatctccccgaatctcaataaaccagtgaacagtgtgactcggcaaaaaaaaaaa

SEQ ID NO: 3 - DNA, 4260 bp, Homo sapiens
cgctccccgctcccgtggctgccgccgccccggggaagaagagacaggggtggggtttgggggaagcgagagaggaggggagagaccctggccaggctggagcctggattcgaggggaggagggacgggaggaggagaaaggtggaggagaagggaggggggagcggggaggagcggccgggcctggggccttgaggcccggggagagccggggagccgggcccgcgcgccgagatgttgctgctgctgctactggcgccactcttcctccgccccccgggcgcgggcggggcgcatacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtgagggtagggtgagggaggacaggccagtagggggagggaaagggagaggggaagggcaggggactcaggaagcagggggtccccatccccagctgggaagaacatgctatccaatctcatctcttgtaaatacatgtccccctgtgagttctgggctgatttgggtctctcatacctctgggaaacagacctttttctctcttactgcttcatgtaattttgtatcacctcttcacaatttagttcgtacctggcttgaagctgctcactgctcacacgctgcctcctcagcagcctcactgcatctttctcttcccatgcaacaccctcttctagttaccacggcaacccctgcagctcctctgcctttgtgctctgttcctgtccagcaggggtctcccaacaagtgctctttccaccccaaaggggcctctccttttctccactgtcataatctctttccatcttacttgcccttctatactttctcacatgtggctccccctgaattttgcttcctttgggagctcattcttttcgccaaggctcacatgctccttgcctctgctctgtgcactcacgctcagcacacatgcatcctcccctctcctgcgtgtgcccactgaacatgctcatgtgtacacacgcttttcccgtatgctttcttcatgttcagtcacatgtgctctcgggtgccctgcattcacagctacgtgtgcccctctcatggtcatgggtctgcccttgagcgtgtttgggtaggcatgtgcaatttgtctagcatgctgagtcatgtctttcctatttgcacacgtccatgtttatccatgtactttccctgtgtaccctccatgtaccttgtgtactttcttcccttaaatcatggtattcttctgacagagccatatgtaccctaccctgcacattgttatgcacttttccccaattcatgtttggtggggccatccacaccctctccttgtcacagaatctccatttctgctcagattccccccatctccattgcattcatgtactaccctcagtctacactcacaatcatcttctcccaagactgctcccttttgttttgtgtttttttgaggggaattaaggaaaaataagtgggggcaggtttggagagctgcttccagtggatagttgatgagaatcctgaccaaaggaaggcacccttgactgttgggatagacagatggacctatggggtgggaggtggtgtccctttcacactgtggtgtctcttggggaaggatctccccgaatctcaataaaccagtgaacagtgtgactcggcaaaaaaaaaaaa

SEQ ID NO: 4 - DNA, 4294 bp, Homo sapiens
cgctccccgctcccgtggctgccgccgccccggggaagaagagacaggggtggggtttgggggaagcgagagaggaggggagagaccctggccaggctggagcctggattcgaggggaggagggacgggaggaggagaaaggtggaggagaagggaggggggagcggggaggagcggccgggcctggggccttgaggcccggggagagccggggagccgggcccgcgcgccgagatgttgctgctgctgctactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagatcgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcagccgttccggcgtgcgcctggaggacttcaactacaacaaccagaccattaccgaccaaatctaccgggcaatgaactcctcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattgttatatccagaactcacagcccaacctgacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtgagggtagggtgagggaggacaggccagtagggggagggaaagggagaggggaagggcaggggactcaggaagcagggggtccccatccccagctgggaagaacatgctatccaatctcatctcttgtaaatacatgtccccctgtgagttctgggctgatttgggtctctcatacctctgggaaacagacctttttctctcttactgcttcatgtaattttgtatcacctcttcacaatttagttcgtacctggcttgaagctgctcactgctcacacgctgcctcctcagcagcctcactgcatctttctcttcccatgcaacaccctcttctagttaccacggcaacccctgcagctcctctgcctttgtgctctgttcctgtccagcaggggtctcccaacaagtgctctttccaccccaaaggggcctctccttttctccactgtcataatctctttccatcttacttgcccttctatactttctcacatgtggctccccctgaattttgcttcctttgggagctcattcttttcgccaaggctcacatgctccttgcctctgctctgtgcactcacgctcagcacacatgcatcctcccctctcctgcgtgtgcccactgaacatgctcatgtgtacacacgcttttcccgtatgctttcttcatgttcagtcacatgtgctctcgggtgccctgcattcacagctacgtgtgcccctctcatggtcatgggtctgcccttgagcgtgtttgggtaggcatgtgcaatttgtctagcatgctgagtcatgtctttcctatttgcacacgtccatgtttatccatgtactttccctgtgtaccctccatgtaccttgtgtactttcttcccttaaatcatggtattcttctgacagagccatatgtaccctaccctgcacattgttatgcacttttccccaattcatgtttggtggggccatccacaccctctccttgtcacagaatctccatttctgctcagattccccccatctccattgcattcatgtactaccctcagtctacactcacaatcatcttctcccaagactgctcccttttgttttgtgtttttttgaggggaattaaggaaaaataagtgggggcaggtttggagagctgcttccagtggatagttgatgagaatcctgaccaaaggaaggcacccttgactgttgggatagacagatggacctatggggtgggaggtggtgtccctttcacactgtggtgtctcttggggaaggatctccccgaatctcaataaaccagtgaacagtgtgactcggcaaaaaaaaaaaa

SEQ ID NO: 5 - DNA, 5786 bp, Homo sapiens
gccgcccctgagcagcctcgccttcgcctcccgcgtttcctgccgtccgccctcccccggccgagctccaggggctgccgcctagcagctcccggcgggagagcggttcagagcgcgcacgggggcgggcggaggcggcccggtgcggggcggccgcgctggagagaggcgcgcgcggagacgccggcccccctctccgcgtttgctcgcttgctccccgcctcccgcactccgctcgctcccaccccttcccggcgtgattgatccgtcacgggcgccgccgctgccgccgccgccgcggccgttctgagccgagccggaaccctagcccgagacggagccggggcccgggccggcgccattgcgcgggcgccgcgggaagaccttggcgcggggcggcgggccgggccaggccatgcgggccgagtgagccggcgcccgcagcccgcggcgcggcatggcttccccgcggagctccgggcagcccgggccgccgccgccgccgccaccgccgcccgcgcgcctgctactgctactgctgctgccgctgctgctgcctctggcgcccggggcctggggctgggcgcggggcgccccccggccgccgcccagcagcccgccgctctccatcatgggcctcatgccgctcaccaaggaggtggccaagggcagcatcgggcgcggtgtgctccccgccgtggaactggccatcgagcagatccgcaacgagtcactcctgcgcccctacttcctcgacctgcggctctatgacacggagtgcgacaacgcaaaagggttgaaagccttctacgatgcaataaaatacgggccgaaccacttgatggtgtttggaggcgtctgtccatccgtcacatccatcattgcagagtccctccaaggctggaatctggtgcagctttcttttgctgcaaccacgcctgttctagccgataagaaaaaatacccttatttctttcggaccgtcccatcagacaatgcggtgaatccagccattctgaagttgctcaagcactaccagtggaagcgcgtgggcacgctgacgcaagacgttcagaggttctctgaggtgcggaatgacctgactggagttctgtatggcgaggacattgagatttcagacaccgagagcttctccaacgatccctgtaccagtgtcaaaaagctgaaggggaatgatgtgcggatcatccttggccagtttgaccagaatatggcagcaaaagtgttctgttgtgcatacgaggagaacatgtatggtagtaaatatcagtggatcattccgggctggtacgagccttcttggtgggagcaggtgcacacggaagccaactcatcccgctgcctccggaagaatctgcttgctgccatggagggctacattggcgtggatttcgagcccctgagctccaagcagatcaagaccatctcaggaaagactccacagcagtatgagagagagtacaacaacaagcggtcaggcgtggggcccagcaagttccacgggtacgcctacgatggcatctgggtcatcgccaagacactgcagagggccatggagacactgcatgccagcagccggcaccagcggatccaggacttcaactacacggaccacacgctgggcaggatcatcctcaatgccatgaacgagaccaacttcttcggggtcacgggtcaagttgtattccggaatggggagagaatggggaccattaaatttactcaatttcaagacagcagggaggtgaaggtgggagagtacaacgctgtggccgacacactggagatcatcaatgacaccatcaggttccaaggatccgaaccaccaaaagacaagaccatcatcctggagcagctgcggaagatctccctacctctctacagcatcctctctgccctcaccatcctcgggatgatcatggccagtgcttttctcttcttcaacatcaagaaccggaatcagaagctcataaagatgtcgagtccatacatgaacaaccttatcatccttggagggatgctctcctatgcttccatatttctctttggccttgatggatcctttgtctctgaaaagacctttgaaacactttgcaccgtcaggacctggattctcaccgtgggctacacgaccgcttttggggccatgtttgcaaagacctggagagtccacgccatcttcaaaaatgtgaaaatgaagaagaagatcatcaaggaccagaaactgcttgtgatcgtggggggcatgctgctgatcgacctgtgtatcctgatctgctggcaggctgtggaccccctgcgaaggacagtggagaagtacagcatggagccggacccagcaggacgggatatctccatccgccctctcctggagcactgtgagaacacccatatgaccatctggcttggcatcgtctatgcctacaagggacttctcatgttgttcggttgtttcttagcttgggagacccgcaacgtcagcatccccgcactcaacgacagcaagtacatcgggatgagtgtctacaacgtggggatcatgtgcatcatcggggccgctgtctccttcctgacccgggaccagcccaatgtgcagttctgcatcgtggctctggtcatcatcttctgcagcaccatcaccctctgcctggtattcgtgccgaagctcatcaccctgagaacaaacccagatgcagcaacgcagaacaggcgattccagttcactcagaatcagaagaaagaagattctaaaacgtccacctcggtcaccagtgtgaaccaagccagcacatcccgcctggagggcctacagtcagaaaaccatcgcctgcgaatgaagatcacagagctggataaagacttggaagaggtcaccatgcagctgcaggacacaccagaaaagaccacctacattaaacagaaccactaccaagagctcaatgacatcctcaacctgggaaacttcactgagagcacagatggaggaaaggccattttaaaaaatcacctcgatcaaaatccccagctacagtggaacacaacagagccctctcgaacatgcaaagatcctatagaagatataaactctccagaacacatccagcgtcggctgtccctccagctccccatcctccaccacgcctacctcccatccatcggaggcgtggacgccagctgtgtcagcccctgcgtcagccccaccgccagcccccgccacagacatgtgccaccctccttccgagtcatggtctcgggcctgtaagggtgggaggcctgggcccggggcctcccccgtgacagaaccacactgggcagaggggtctgctgcagaaacactgtcggctctggctgcggagaagctgggcaccatggctggcctctcaggaccactcggatggcactcaggtggacaggacggggcagggggagacttggcacctgacctcgagccttatttgtgaagtccttatttcttcacaaagaagaggaacggaaatgggacgtcttccttaacatctgcaaacaaggaggcgctgggatatcaaacttgcaaaaaaaaaaaaaaaacaaaacaaaaaaaaaaactagacaaggagagaggcactagaactccagctggaagtcacggagtggctcgagcagccttgggaagaggcaaggagcttctgaagaaactgcctctgcacacacatcactggctgtgacccctcaggctagcccttctccactctgggggaggaggtgggaagggccaccaggcccccagctgccaggccagctgaccccagccttcctggaacagggagtctgcaggagcgcagacaggcacagccctggagcaggcaggccgagggctgcggcactggagcaggctgacttacatgctccacatgggacctgtgtcacccaatgagatgtttgttactctggtaaatgccacacgttaacacaataacacccattcctgggaccgtggggatttagggcacgtcactgcagacacgctctgcagcattcaccgacagtctgtcatgcacccaccacgttggccacgtccttgtgttcctatcggatgctcccagtaaccagggggaccacccgagctaatcatggaatgtctgttcccagcaaacacgataaagaaagattgtgcactttaacctctctcatcagggcccaagggctggctgggattttttttttttttttttcccactaactttgtttctgaccaaagtgaattggaggcattctgctaaaagacatccccgtagacataggggagagagttgctggctgagggcttcccttggcttccagaaggcagccttccatccagacaagccagtgagctctccccttgggatcactggggtgatcagtcagcagattgattctcattcataagatcattcctccctttaaattgagcccctaagagcactggcctgggagtcagacagacctgggttcaagtcctcagtcccctgcccactccctacgtgactttgatcaggtcactagtgtctctctgagcctcagtttcccctctgtaacttggggttgaactaaaacacctgtcctgcctacctcacaaggtcactctgaggattgaaacttgatcttgtccaggaaagctttgtaccaaacagtgaagccgccctgatccgtgaggtatgagtatgactctgaccttcagccctccctacagccgggggtgtggcccagagaagcttccagcacagccctctacccagaacatccgggctggagggaggctcccagtgacttttctgacattcctagacaggttcattctttgctcaagaaaggcctgaatgacaatgtccaggatgtctgcacaactgagcagctcgctcactccctaaagaaacctattggcagcttcaacaggcaggcaataatctcttcccagaaccactgcagtcaggaataaactgttttctccaccaggctttgacaaaagggcccacaggaatcttaccaatgccaacatttcaaagcaccctatttcacgtagcatagctttctgctccccttccccaaagagaggttatggaggtactgtagcttttagggaaaaaaaaaatgttaacacatcacaggtcaagttgaagtcattctctgtttaggcactaaaaatcggtgttgtcactcactgtgtattaccagtatttacttgctttcttgatttcaccaaaaccaaatttaatttaaaggaccacattaatttttcaaagggaaagagacaattaattgtacataatgtatacacacacaaaaaaaaaaatacctgtagaaatattattccagcatagcaggaaaacaaacaaaagtattggactgtcggaggtgagcctgtgcgtctgtaaccctttgtgactcctgagcgtgcgctgtcttctaggttaactcacgaagtacattctctgtcttactgatactgtaggttcacccatttttttttaatttcctcgcaaataacaagacccacagaagtgactctagctacttaatggttctgttcttttatatgcagcaaacacaccgtccatttctgaagaggcttcggcctgaaggcattttccaatgatgttagtgcacaaaacgctttaaattagactggaactgccagaatcaaatgtaaatgaggaatttctcgtacccctactgcatggtatcgatttttaataaattgttgcaaatttgtttttatgaataaaaggaaaaaacctgtcgtcttt

(19) United States
(12) Patent Application Publication
(10) Pub. No.: US 2009/0041806 A1
(43) Pub. Date: Feb. 12, 2009

(54) USE OF PRODRUGS OF GABA ANALOGS, ANTISPASTICITY AGENTS, AND PRODRUGS OF GABA B RECEPTOR AGONISTS FOR TREATING SPASTICITY

(75) Inventor: Kenneth C. CUNDY, Redwood City, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: XenoPort, Inc., Santa Clara, CA (US)

(21) Appl. No.: 12/139,057

(22) Filed: Jun. 13, 2008

Related U.S. Application Data
(60) Provisional application No. 60/944,475, filed on Jun. 15, 2007.

(51) Int. Cl. A61K 39/08; A61K 31/27

(52) U.S. Cl. 424/239.1; 514/484; 514/478

(57) ABSTRACT

Methods of treating spasticity by administering a colonically absorbable prodrug of a GABA analog having antispastic activity that is not directly mediated by the GABA_{B} receptor, optionally in combination with an antispasticity agent or a colonically absorbable prodrug of a GABA_{B} receptor agonist are disclosed. In particular, methods of treating spasticity by administering a colonically absorbable prodrug of gabapentin or a colonically absorbable prodrug of pregabalin, in combination with a colonically absorbable prodrug of R-baclofen are disclosed.

This application claims the benefit under 35 U.S.C. § 119(e) of U.S. Provisional Application Ser. No. 60/944,475 filed Jun. 15, 2007, which is incorporated by referenced herein in its entirety.

### FIELD

Methods of treating spasticity are disclosed wherein a colonically absorbable prodrug of a GABA analog having antispastic activity that is not directly mediated by the GABA_{B} receptor, optionally in combination with an antispasticity agent or a colonically absorbable prodrug of a GABA_{B} receptor agonist, is administered to a patient in need of such treatment. In particular, methods of treating spasticity are disclosed in which a colonically absorbable prodrug of gabapentin or a colonically absorbable prodrug of pregabalin, in combination with a colonically absorbable prodrug of R-baclofen, is administered to a patient in need of such treatment.

### BACKGROUND

Baclofen (R,S-baclofen), (±)-4-amino-3-(4-chlorophenyl)butanoic acid, (1):

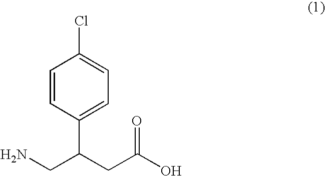

is a GABA_{B} receptor agonist that has been used in the United States since 1977 for alleviating the signs and symptoms of spasticity resulting from multiple sclerosis or spinal cord injury. The mechanism of action of baclofen in spasticity appears to involve agonism at GABA_{B} receptors of the spinal cord (Price et al., Nature 1984, 307(5946), 71-4). Baclofen is believed to inhibit the transmission of both monosynaptic and polysynaptic reflexes at the spinal cord level, possibly by hyperpolarization of primary afferent fiber terminals, with resultant relief of muscle spasticity. Baclofen was approved for marketing as a racemic compound, however preclinical studies have since demonstrated that the antispasticity activity of the drug resides exclusively in the R-isomer (Albright et al., Neurology, 1995, 45(11), 2110-2111). The active isomer (R-baclofen) has also been studied in several clinical trials for the treatment of trigeminal neuralgia, affective disorder, and cerebral spasticity.

Baclofen has a number of significant pharmacokinetic limitations including a narrow window of absorption in the upper small intestine and rapid clearance from the blood. Consequently baclofen is taken three to four times per day to maintain the therapeutic effects. (3R)-4-{[(1S)-2-Methyl-1-(2-methylpropanoyloxy)propoxy]carbonylamino}-3-(4-chlorophenyl)butanoic acid is an example of a prodrug of R-baclofen that was designed to overcome the pharmacokinetic deficiencies of baclofen (Gallop et al., U.S. Pat. Nos. 7,109,239 and 7,227,028, each of which is incorporated by reference herein in its entirety). (3R)-4-{[(1S)-2-Methyl-1-(2-methylpropanoyloxy)propoxy]carbonylamino}-3-(4-chlorophenyl)butanoic acid is engineered to take advantage of absorption pathways present throughout the intestinal tract. In preclinical species, (3R)-4-{[(1S)-2-methyl-1-(2-methylpropanoyloxy)propoxy]carbonylamino}-3-(4-chlorophenyl)butanoic acid is well absorbed in the small and large intestine and undergoes rapid metabolism to R-baclofen following absorption. The improved colonic absorption of (3R)-4-{[(1S)-2-methyl-1-(2-methylpropanoyloxy)propoxy]carbonylamino}-3-(4-chlorophenyl)butanoic acid allows development of a controlled release formulation with reduced dosing frequency. Reducing peak baclofen blood levels may also decrease side effects. Therefore, (3R)-4-{[(1S)-2-methyl-1-(2-methylpropanoyloxy)propoxy]carbonylamino}-3-(4-chlorophenyl)butanoic acid in a controlled release formulation offers the potential of a pharmacologically novel treatment for spasticity with an improved safety profile and greater patient convenience compared to baclofen.

The γ-aminobutyric acid (γ-aminobutyric acid is abbreviated herein as GABA) analog, gabapentin, (2):

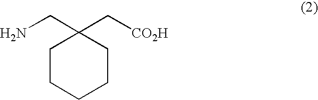

Gabapentin has shown activity in placebo-controlled, double-blind clinical studies of spasticity (Priebe et al., Spinal Cord 1997, 35(3), 171-175; and Gurenthal et al., Spinal Cord 1997, 35(10), 686-689). The mechanism of action of gabapentin in treating spasticity is unknown. However, gabapentin is a GABA analog that does not interact significantly with GABA_{B} receptors (Schlicker et al., Arzneimittelforschung 1985, 35(9), 1347-9). It has recently been demonstrated that the mechanism of action of gabapentin in spasticity differs from that of baclofen (Shimizu et al., J Pharmacol Sci. 2004, 96(4), 444-449). This is consistent with reports that the binding of gabapentin in rat brain is not inhibited by baclofen (Suman-Chauhan et al., Eur J Pharmacol 1993, 244(3), 293-301).

Like baclofen, gabapentin has a number of significant pharmacokinetic limitations including a narrow window of absorption in the upper small intestine and rapid clearance from the blood. These limitations require gabapentin to be administered three to four times per day to maintain therapeutic effects. 1-{[(α-Isobutanoyloxyethoxy)carbonyl]aminomethyl}-1-cyclohexane acetic acid is an example of a prodrug of gabapentin that is designed to overcome the pharmacokinetic deficiencies of gabapentin (see Zerangue, U.S. Publication No. 2003/0158254; Gallop et al., U.S. Application Publication No. 2003/0158089, and U.S. Pat. Nos. 6,955,888 and 7,053,076; and International Publication Nos. WO 02/100172, WO 02/100392, WO 02/100347, WO 02/100344, WO 02/42414, WO 02/28881, WO 02/28882, WO 02/44324, WO 02/32376, WO 02/28883, and WO 02/28411; Cundy et al., J Pharm Exptl Therapeutics 2004, 311(1), 315-23; Cundy et al., J Pharm Exptl Therapeutics 2004, 311(1), 324-33; Canafax et al., 58^{th} Annual Meeting of the American Academy of Neurology (AAN), San Diego, Calif., Apr. 1-8, 2006; Fenney et al., 58^{th} Annual Meeting of the American Academy of Neurology (AAN), San Diego, Calif., Apr. 1-8, 2006; each of which is incorporated by reference herein in its entirety). 1-{[(α-Isobutanoyloxyethoxy)carbonyl]aminomethyl}-1-cyclohexane acetic acid was engineered to take advantage of absorption pathways present throughout the intestinal tract. In preclinical species, 1-{[(α-isobutanoyloxyethoxy)carbonyl]aminomethyl}-1-cyclohexane acetic acid is well absorbed in the small and large intestine and undergoes rapid metabolism to gabapentin following absorption. The improved colonic absorption of 1-{[(α-isobutanoyloxyethoxy)carbonyl]aminomethyl}-1-cyclohexane acetic acid potentially allows development of a controlled release formulation of gabapentin with reduced dosing frequency. Reducing the peak/trough ratio of gabapentin blood levels may decrease side effects and maximize duration of therapy. Therefore, prodrugs of gabapentin such as 1-{[(α-isobutanoyloxyethoxy)carbonyl]aminomethyl}-1-cyclohexane acetic acid administered in a controlled release formulation offer the potential of pharmacologically novel treatments for spasticity, with an improved safety profile, and greater patient convenience compared to gabapentin.

Pregabalin, (S)-(3-aminomethyl)(3S)-5-methylhexanoic acid, (3):

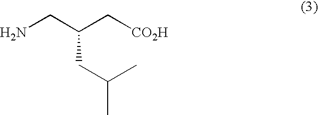

another GABA analog, has been approved in the United States as an anticonvulsant (see Bryans et al., J. Med. Chem. 1998, 41, 1838-1845). Like gabapentin, pregabalin is not significantly absorbed from the large intestine but rather is absorbed in the small intestine via the large neutral amino acid transporter (Jezyk et al., Pharm Res. 1999, 16, 519-526). Colonically absorbable prodrug strategies for pregabalin have been demonstrated (see e.g., Gallop et al., U.S. Pat. No. 6,818,787 and Yao and Gallop, U.S. Application Ser. Nos. 61/023,808 and 61/023,813 filed Jan. 25, 2008, each of which is incorporated by reference herein in its entirety). Pregabalin as well as gabapentin may interact with the α2δ subunit of calcium channel modulator to exert their respective pharmacologic effects (Gee et al., J Biol Chem 1996, 2771, 5768-5776; and Bryans et al., Med Res Rev, 1999, 19, 149-177).

The antispastic activity of GABA analogs such as gabapentin and pregabalin and GABA_{B} receptor agonists such as baclofen appears to be mediated by different mechanisms. Additional and/or combined mechanisms of action have been proposed for other compounds exhibiting antispasticic activity such as allosteric modulation of the GABA_{B} receptor and/or as modulators of the α2δ subunit of calcium channel modulator. Combinations of compounds having antispastic activity acting through different mechanisms are expected to have synergistic effects useful in treating spasticity. For example, colonically absorbable prodrugs of GABA analogs having antispastic activity that is not directly mediated by the GABA_{B} receptor, optionally in combination with an antispasticity agent or a colonically absorbable prodrug of a GABA_{B} receptor agonist can provide enhanced efficacy in treating spasticity.

### SUMMARY

In a first aspect, methods of treating spasticity in a patient are disclosed comprising administering to a patient in need of such treatment a therapeutically effective amount of a colonically absorbable prodrug of a GABA analog having antispastic activity that is not directly mediated by the GABA_{B} receptor.

In a second aspect, methods of treating spasticity in a patient are disclosed comprising administering to a patient in need of such treatment a therapeutically effective amount of a colonically absorbable prodrug of a GABA analog having antispastic activity that is not directly mediated by the GABA_{B} receptor, and an antispasticity agent.

In a third aspect, methods of treating spasticity in a patient are disclosed comprising administering to a patient in need of such treatment a therapeutically effective amount of a colonically absorbable prodrug of a GABA analog having antispastic activity that is not directly mediated by the GABA_{B} receptor, and a colonically absorbable prodrug of a GABA_{B} receptor agonist.

### DETAILED DESCRIPTION

### Definitions

A dash (“-”) that is not between two letters or symbols is used to indicate a point of attachment for a moiety or substituent. For example, —CONH₂ is attached through the carbon atom.

“Alkyl” by itself or as part of another substituent refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group can have from 1 to 20 carbon atoms, in certain embodiments, from 1 to 10 carbon atoms, in certain embodiments, from 1 to 6 carbon atoms, and in certain embodiments, from 1 to 3 carbon atoms.

“Acyl” by itself or as part of another substituent refers to a radical —C(O)R³⁰, where R³⁰ is chosen from hydrogen, alkyl, cycloalkyl, heterocycloalkyl, aryl, arylalkyl, heteroalkyl, heteroaryl, and heteroarylalkyl, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR³¹ where R³¹ is chosen from alkyl, cycloalkyl, cycloalkylalkyl, aryl, and arylalkyl, as defined herein. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)OR³² where R³² represents an alkyl, as defined herein. Examples of alkoxycarbonyl groups include, but are not limited to, methoxycarbonyl, ethoxycarbonyl, propoxycarbonyl, and butoxycarbonyl, and the like.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocylic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms, from 6 to 12 carbon atoms, and in certain embodiments, from 6 to 8 carbon atoms. Aryl, however, does not encompass or overlap in any way with heteroaryl, separately defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“Aryldialkylsilyl” by itself or as part of another substituent refers to the radical —SiR³²R³³R³⁴ where one of R³², R³³ or R³⁴ is aryl as defined herein and the other two of R³², R³³ or R³⁴ are alkyl as defined herein.

“AUC” is the area under a curve representing the concentration of a compound or metabolite thereof in a biological fluid in a patient as a function of time following administration of the compound to the patient. In certain embodiments, the compound can be a prodrug and the metabolite can be a drug. Examples of biological fluids include plasma and blood. The AUC may be determined by measuring the concentration of a compound or metabolite thereof in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the present disclosure, an AUC for a GABA analog or metabolite thereof may be determined by measuring the concentration of the GABA analog or metabolite thereof in the plasma or blood of a patient following administration of a colonically absorbable prodrug of a GABA analog to the patient.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to maximum concentration (Tₘₐₓ), and the maximum drug concentration (Cₘₐₓ), where Cₘₐₓis the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Cₘₐₓ” is the maximum concentration of a drug in the plasma or blood of a patient following administration of a dose of the drug or prodrug thereof to the patient.

“Tₘₐₓ” is the time to the maximum (peak) concentration (Cₘₐₓ) of a drug in the plasma or blood of a patient following administration of a dose of the drug or prodrug thereof to the patient.

“Carbamoyl” by itself or as part of another substituent refers to the radical —C(O)NR³⁹R⁴⁰ where R³⁹ and R⁴⁰ are independently hydrogen, alkyl, cycloalkyl or aryl, as defined herein.

“Colonically absorbable prodrug of a GABA analog” means a prodrug of a GABA analog, as defined herein, which provides an AUC of the corresponding GABA analog following colonic administration of the prodrug that is at least two times greater than the AUC of the GABA analog following colonic administration of an equivalent amount of the GABA analog itself.

“Colonically absorbable prodrug of a GABA_{B} receptor agonist” means a prodrug of a GABA_{B} receptor agonist, as defined herein, which provides an AUC of the corresponding GABA_{B} receptor agonist following colonic administration of the prodrug that is at least two times greater than the AUC of the GABA_{B} receptor agonist following colonic administration of an equivalent amount of the GABA_{B} receptor agonist itself.

“Compounds” of Formula (I), Formula (II), Formula (III), and Formula (IV) disclosed herein, include any specific compounds within these formulae. Compounds may be identified either by their chemical structure and/or chemical name. When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds of Formula (I), Formula (II), Formula (III), and Formula (IV) include, but are not limited to, optical isomers of compounds of Formula (I), Formula (II), Formula (III), and Formula (IV), racemates thereof, and other mixtures thereof. In such embodiments, the single enantiomers or diastereomers, i.e., optically active forms, can be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column. In addition, compounds of Formula (I), Formula (II), Formula (III), and Formula (IV) include Z- and E-forms (or cis- and trans-forms) of compounds with double bonds.

Compounds of Formula (I), Formula (II), Formula (III), and Formula (IV) may also exist in several tautomeric forms including the enol form, the keto form, and mixtures thereof. Accordingly, the chemical structures depicted herein encompass all possible tautomeric forms of the illustrated compounds. Compounds of Formula (I), Formula (II), Formula (III), and Formula (IV) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Certain compounds may exist in multiple crystalline or amorphous forms. Compounds of Formula (I), Formula (II), Formula (III), and Formula (IV) include pharmaceutically acceptable salts thereof, or pharmaceutically acceptable solvates of the free acid form of any of the foregoing, as well as crystalline forms of any of the foregoing.

Further, when partial structures of the compounds are illustrated, an asterisk (*) indicates the point of attachment of the partial structure to the rest of the molecule.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₅₋₁₂ cycloalkyl, and in certain embodiments, C₃₋₇ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is Cl₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“GABA analog” means a compound having the following structure:

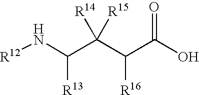

wherein:

R¹² is hydrogen, or R¹² and R¹⁶ together with the atoms to which they are bonded form a ring chosen from an azetidine, substituted azetidine, pyrrolidine, and substituted pyrrolidine ring;

R¹³ and R¹⁶ are independently chosen from hydrogen, alkyl, substituted alkyl, aryl, substituted aryl, arylalkyl, substituted arylalkyl, cycloalkyl, substituted cycloalkyl, heteroalkyl, substituted heteroalkyl, heterocycloalkyl, substituted heterocycloalkyl, heteroaryl, substituted heteroaryl, heteroarylalkyl, and substituted heteroarylalkyl; and

R¹⁴ and R¹⁵ are independently chosen from hydrogen, alkyl, substituted alkyl, acyl, substituted acyl, aryl, substituted aryl, arylalkyl, substituted arylalkyl, cycloalkyl, substituted cycloalkyl, heteroalkyl, substituted heteroalkyl, heterocycloalkyl, substituted heterocycloalkyl, heteroaryl, substituted heteroaryl, heteroarylalkyl, and substituted heteroarylalkyl; or R¹⁴ and R¹⁵ together with the carbon atom to which they are bonded form a ring chosen from a cycloalkyl, substituted cycloalkyl, heterocycloalkyl, substituted heterocycloalkyl, and bridged cycloalkyl ring.

In certain embodiments of a GABA analog, each substitute group is independently chosen from halogen, —NH₂, —OH, —CN, —COOH, —C(O)NH₂, —C(O)OR¹⁰, and —NR¹⁰₃⁺ wherein each R¹⁰ is independently C₁₋₃ alkyl.

In certain embodiments of a GABA analog, R¹² is hydrogen.

In certain embodiments of a GABA analog, R¹² is hydrogen, R¹³ is hydrogen, R¹⁶ is hydrogen, and R¹⁴ and R¹⁵ together with the carbon atom to which they are bonded form a cyclohexyl ring.

In certain embodiments of a GABA analog, R¹² is hydrogen, R¹³ is hydrogen, R¹⁶ is hydrogen, R¹⁴ is hydrogen, and R¹⁵ is isobutyl.

In certain embodiments, a GABA analog is chosen from gabapentin and pregabalin. Furthermore, a number of GABA analogs with considerable pharmaceutical activity have been synthesized in the art (see, e.g., Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO 92/09560; Silverman et al., International Publication No. WO 93/23383; Horwell et al., International Publication No. WO 97/29101, Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 00/15611; Bryans, International Publication No. WO 00/31020; Bryans et al., International Publication No. WO 00/50027; and Bryans et al., International Publication No. WO 02/00209); International Publication No. WO 98/23383; Bryans et al., J. Med. Chem. 1998, 41, 1838-1845; Bryans et al., Med. Res. Rev. 1999, 19, 149-177, U.S. Application Publication No. 2002/0111338; International Publication No. WO 99/08670; International Publication No. WO 99/21824; U.S. Patent Ser. No. 60/160,725; UK Patent No. GB 2 374 595). Pharmaceutically important GABA analogs include, for example, gabapentin, pregabalin, vigabatrin, and baclofen.

“GABA analog having antispastic activity that is not directly mediated by the GABA_{B} receptor” means that the antispastic activity exhibited by the GABA analog is not associated with binding of the GABA analog to the GABA_{B} receptor and/or the GABA analog does not affect biochemical pathways associated with the agonist-GABA_{B} receptor complex to elicit the antispastic activity such as the replacement of GDP with an activating guanidine triphosphate allowing for the G-coupled protein to interact with a membrane bound effector site, or causing membrane depolarization through voltage-gated calcium channel influx restriction into the presynaptic terminal and postsynaptic binding, which increases receptor operated potassium channel conductance (see e.g., Francisco et al., Phys Med Rehab Clin NA, 2001, 12(4), 875-888). Mediated includes direct agonist activity, antagonist activity, and allosteric modulation of the GABA_{B} receptor. The ability of a GABA analog to directly interact with the GABA_{B} receptor can be determined using, for example, any of the functional assays described in Examples 1-3. In these assays, a GABA analog that does not directly interact with the GABA_{B} receptor, e.g., its effect is not directly mediated by the GABA_{B} receptor, is identified by a negative result. Antispastic activity can be determined based on efficacy studies using animal models and/or in clinical trials.

“GABA_{B} receptor” includes the subtypes of presynaptic receptors comprising heteroreceptors as well as autoreceptors, and postsynaptic receptors that are inhibited by GABA and are coupled through G-proteins to Ca²⁺ or K⁺ channels (see Kerr and Ong, Pharmacol Ther 1995, 67(2), 187-246). The GABA_{B} receptor exists as a heterodimer with two subunits, GABA_{B1} and GABA_{B2}, which provide different functions but are mutually dependent (see Bowery et al., Pharmcol Rev 2002, 54, 247-264; and Bowery et al., Current Opin. Pharmacol 2006, 6, 37-43). The GABA_{B1} subunit contains the GABA-binding domain and the GABA_{B2} subunit provides the G-protein-coupling mechanism and also incorporates an allosteric modulatory site within its heptahelical structure. Four different functional isoforms of the human GABA_{B1} subunit have been identified; however, there is no unequivocal evidence for distinct GABA_{B} receptor subtypes. The variants of the GABA_{B1} subunit do not appear to have significant pharmacological differences with respect to activator or inhibitor binding. The human GABA_{B1} receptor variant proteins may be encoded by the nucleotide sequences set forth as SEQ ID NO: 1 (GABA_{B1}, NM_{—}001470); SEQ ID NO: 2 (GABA_{B2}, NM_{—}021903); SEQ ID NO: 3 (GABA_{B3}, NM_{—}021904); or SEQ ID NO: 4 (GABA_{B4}, NM_{—}021905); and the amino acid sequence of human GABA_{B21} receptor is encoded by the nucleotide sequence set forth as SEQ ID NO: 5 (GABA_{B2}, NM_{—}005458). Reference to a GABA_{B} receptor includes the amino acid sequence described in or encoded by the nucleotides comprising the sequences set forth as SEQ ID NO:1, SEQ ID NO:2, SEQ ID NO:3, or SEQ ID NO:4, the sequences set forth with the above-identified GenBank reference numbers, and allelic, cognate and induced variants and fragments thereof retaining essentially the same activity. Usually such variants show at least 90% sequence identity to the exemplary GenBank nucleic acid or amino acid sequence.

“GABA_{B} receptor agonist” means baclofen and compounds that elicit a positive effect in any of the functional assays described herein, for example, in Examples 1-3, or in any other accepted functional assay for determining GABA_{B} receptor agonist activity known in the art.

“Halogen” refers to a fluoro, chloro, bromo, or iodo group.

“Heteroalkyl” by itself or as part of another substituent refers to an alkyl group in which one or more of the carbon atoms (and any associated hydrogen atoms) are independently replaced with the same or different heteroatomic groups. Examples of heteroatomic groups include, but are not limited to, —O—, —S—, —O—O, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —SO—, —SO₂—, —SnR⁴³R⁴⁴—, and the like, where R³⁷, R³⁸, R³⁹, R⁴⁰, R⁴¹, R⁴², R⁴³, and R⁴⁴ are independently chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkanyl,” “heteroalkenyl,” or “heteroalkynyl” is used. In certain embodiments, R³⁷, R³⁸, R³⁹, R⁴⁰, R⁴¹, R⁴², R⁴³, and R⁴⁴ are independently chosen from hydrogen, C₁₋₅ alkyl and substituted C₁₋₅ alkyl. In certain embodiments, heteroalkyl comprises one or more heteroatoms chosen from O and N.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heteroaromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 10-membered heteroaryl. In certain embodiments heteroaryl groups are those derived from thiophene, pyrrole, benzothiophene, benzofuran, indole, pyridine, quinoline, imidazole, oxazole, or pyrazine

“Heteroarylalkyl” by itself or as part of another substituent refers to an acyclic alkyl radical in which one of the hydrogen atoms bonded to a carbon atom, is replaced with a heteroaryl group. Typically a terminal or sp³ carbon atom is the atom replaced with the heteroaryl group. Where specific alkyl moieties are intended, the nomenclature “heteroarylalkanyl,” “heteroarylalkenyl,” and “heterorylalkynyl” is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

Heterocycloalkyl” by itself or as part of another substituent refers to a saturated, partially unsaturated, or saturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“N-oxide” refers to the zwitterionic nitrogen oxide of a tertiary amine base.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom. Examples of heteroatoms to replace the carbon atoms include, but are not limited to, N, P, O, S, and Si, etc. In certain embodiments, a parent heteroaromatic ring system comprises one or more heteroatoms chosen from N and O. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example, a human.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound is replaced by a metal ion, e.g., an alkali metal ion, an alkaline earth metal ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to at least one colonically absorbable prodrug of a GABA analog having antispastic activity that is not directly mediated by the GABA_{B} receptor, an antispasticity agent, and/or a colonically absorbable prodrug of a GABA_{B} receptor agonist, and at least one pharmaceutically acceptable vehicle, with which the at least one colonically absorbable prodrug of a GABA analog having antispastic activity that is not directly mediated by the GABA_{B} receptor, an antispasticity agent, and/or a colonically absorbable prodrug of a GABA_{B} receptor agonist is administered to a patient.

“Prodrug” refers to a derivative of a drug molecule that requires a transformation within the body to release the active drug. Prodrugs are frequently, although not necessarily, pharmacologically inactive until converted to the parent drug. Prodrugs can be obtained by bonding a promoiety (defined herein) typically via a functional group, to a drug. For example, referring to compounds of Formula (I) or Formula (II), the promoiety is bonded to the drug via the amine functional group of the GABA analog. Compounds of Formula (I) and Formula (II) are colonically absorbable prodrugs of GABA analogs that can be metabolized within a patient's body to release the corresponding GABA analog. Compounds of Formula (II) are colonically absorbable prodrugs of GABA_{B} receptor agonists that can be metabolized within a patient's body to release the corresponding GABA_{B} receptor agonist. Compounds of Formula (IV) are colonically absorbable prodrugs of R-baclofen that can be metabolized within a patient's body to release R-baclofen.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach, or the agent may be supplied exogenously.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules are water.

“Substituted” refers to a group in which one or more hydrogen atoms are independently replaced with the same or different substitute group (s). Examples of substitute groups include, but are not limited to, -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, and —C(NR⁶²)NR⁶⁰R⁶¹ where M is independently a halogen; R⁶⁰, R⁶¹, R⁶², and R⁶³ are independently chosen from hydrogen, alkyl, alkoxy, cycloalkyl, heterocycloalkyl, aryl, and heteroaryl, or R⁶⁰ and R⁶¹ together with the nitrogen atom to which they are bonded form a ring chosen from a heterocycloalkyl ring. In certain embodiments, R⁶⁰, R⁶¹, R⁶², and R⁶³ are independently chosen from hydrogen, C₁₋₆ alkyl, C₁₋₆ alkoxy, C₃₋₁₂ cycloalkyl, C₃₋₁₂ heterocycloalkyl, C₆₋₁₂ aryl, and C₆₋₁₂ heteroaryl. In certain embodiments, a substitute group is independently chosen from halogen, —OH, —CN, —CF₃, ═O, —NO₂, C₁₋₃ alkoxy, C₁₋₃ alkyl, —COOR⁶⁴ wherein R⁶⁴ is chosen from hydrogen and C₁₋₃ alkyl, and —NR⁶⁵₂ wherein each R⁶⁵ is independently chosen from hydrogen and C₁₋₃ alkyl. In certain embodiments, each substitute group is independently chosen from halogen, —OH, —CN, —CF₃, —C(O)NH₂, —COOR¹⁰, and —NR¹⁰₂ wherein each R¹⁰ is independently chosen from hydrogen and C₁₋₃ alkyl.

“Sustained release” refers to release of a compound from a pharmaceutical composition dosage form at a rate effective to achieve a therapeutic or prophylactic concentration of the compound or active metabolite thereof, in the systemic circulation of a patient over a prolonged period of time relative to that achieved by administration of an immediate release formulation of the same compound by the same route of administration. In some embodiments, release of a compound occurs over a time period of at least about 4 hours, such as at least about 8 hours, at least about 12 hours, at least about 16 hours, at least bout 20 hours, and in some embodiments, at least about 24 hours.

“Treating” or “treatment” of a disease or disorder refers to arresting or ameliorating a disease, disorder, or at least one of the clinical symptoms of a disease or disorder; reducing the risk of acquiring a disease, disorder, or at least one of the clinical symptoms of a disease or disorder; slowing or delaying the development of a disease, disorder or at least one of the clinical symptoms of the disease or disorder; and/or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

In certain embodiments, the terms “treating” and “treatment” and “to treat” refer to preventing, reducing, or eliminating spasticity and/or the accompanying symptoms of spasticity in a patient such as for example, painful flexor or extensor spasms, increased or exaggerated deep tendon reflexes, hyperreflexia, loss of dexterity, muscle weakness, exaggerated tendon jerks, and clonus. Treatment of spasticity refers to any indicia of success in prevention, reduction, or elimination or amelioration of spasticity including any objective or subjective parameter such as abatement, remission, diminishing of symptoms, prevention, or lessening of spasticity symptoms or making the condition more tolerable to the patient, making the spasticity less debilitating, or improving a patient's physical or mental well-being.

“Therapeutically effective amount” refers to the amount of a compound that, when administered to a subject for treating a disease or disorder, or at least one of the clinical symptoms of a disease or disorder, is sufficient to effect such treatment of the disease, disorder, or symptom. The “therapeutically effective amount” may vary depending, for example, on the compound, the disease, disorder, and/or symptoms of the disease or disorder; the severity of the disease, disorder, and/or symptoms of the disease or disorder; the age, weight, and/or health of the patient to be treated, and the judgment of the prescribing physician. An appropriate therapeutically effective amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Trialkylsilyl” by itself or as part of another substituent refers to a radical —SiR⁵⁰R⁵¹R⁵² where R⁵⁰, R⁵¹ and R⁵² are independently alkyl as defined herein.

Reference is now be made in detail to certain embodiments of compounds, compositions, and methods. The disclosed embodiments are not intended to be limiting of the claims. To the contrary, the claims are intended to cover all alternatives, modifications, and equivalents.

### GABA Analogs Having Antispastic Activity that is not Directly Mediated by the GABA_{B} Receptor

GABA analogs and methods of synthesizing GABA analogs are known in the art (see e.g., Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO 92/09560; Silverman et al., International Publication No. WO 93/23383; Horwell et al., International Publication No. WO 97/29101; Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 00/15611; Bryans, International Publication No. WO 00/31020; and Bryans et al., International Publication No. WO 00/50027). GABA analogs are also disclosed in Dooley et al., U.S. Pat. No. 7,164,034 and U.S. Application Publication Nos. 2007/0027212 and 2004/0186177; Fraser et al., U.S. Application Publication Nos. 2006/0276542 and 2006/0264509; and Graham et al., U.S. Application Publication No. 2006/0247291).

Certain GABA analogs such as gabapentin and pregabalin are known to have anticonvulsant and/or antispastic activity (Priebe et al., Spinal Cord 1997, 35(3), 171-175; Gurenthal et al., Spinal Cord 1997, 35(10), 686-689; and Bryans et al., J Med Chem 1998, 41, 1838-1845). Antispastic activity of GABA analogs can be determined using, for example, the methods disclosed in the above references based on animal models of spasticity and/or clinical trials. Whether the antispastic activity of a GABA analog is directly mediated by the GABA_{B} receptor can be determined using functional assays such as those described in Examples 1-3 or others known in the art.

### Colonically Absorbable Prodrugs of GABA Analogs

The broad pharmaceutical activities of GABA analogs such as gabapentin (2) and pregabalin (3):

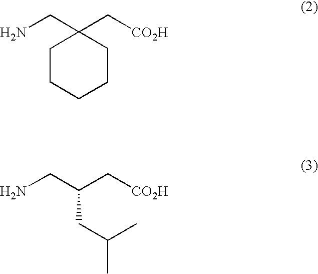

have stimulated intensive interest in preparing related compounds that have superior pharmaceutical properties relative to GABA, e.g., the ability to cross the blood-brain-barrier (see, e.g., Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO 92/09560; Silverman et al., International Publication No. WO 93/23383; Horwell et al., International Publication No. WO 97/29101, Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 00/15611; Belliot et al., International Publication No. WO 00/31020; Bryans et al., International Publication No. WO 00/50027; and Bryans et al., International Publication No. WO 02/00209).

One significant problem associated with the clinical use of many GABA analogs, including gabapentin and pregabalin, is rapid systemic clearance. Consequently, these drugs require frequent dosing to maintain a therapeutic or prophylactic concentration in the systemic circulation (Bryans et al., Med. Res. Rev. 1999, 19, 149-177). For example, dosing regimens of 300-600 mg doses of gabapentin administered three times per day are typically used for anticonvulsive therapy. Higher doses (1800-3600 mg/day in three or four divided doses) are typically used for the treatment of neuropathic pain states. Although oral sustained released formulations are conventionally used to reduce the dosing frequency of drugs that exhibit rapid systemic clearance, oral sustained release formulations of gabapentin and pregabalin have not been developed because these drugs are not absorbed via the large intestine. Rather, these compounds are typically absorbed in the small intestine by one or more amino acid transporters such as the large neutral amino acid transporter (Jezyk et al., Pharm. Res. 1999, 16, 519-526). The limited residence time of both immediate release and sustained release oral dosage forms in the proximal absorptive region of the gastrointestinal tract necessitates frequent daily dosing of oral dosage forms of these drugs, and has prevented the successful application of sustained release technologies to many GABA analogs.

One method for overcoming rapid systemic clearance of GABA analogs is to administer an extended release dosage formulation containing a colonically absorbed GABA analog prodrug (Gallop et al., U.S. Pat. Nos. 6,818,787, 6,972,341, 7,026,351, and 7,060,727; and U.S. Published Application Nos. 2005/0222431 and 2006/0122125; and Estrada et al., 2005/0154057; and International Publication Nos. WO 02/100347 and WO 02/100349; each of which is incorporated by reference herein in its entirety). Sustained release formulations enable a colonically absorbed GABA analog prodrug to be absorbed over a wider region of the gastrointestinal tract than the parent drug including across the wall of the colon where sustained release oral dosage forms typically spend a significant portion of gastrointestinal transit time. These prodrugs are typically converted to the parent GABA analog upon absorption in vivo.

In certain embodiments, a colonically absorbable prodrug of a GABA analog having antispastic activity that is not directly mediated by the GABA_{B} receptor is chosen from a colonically absorbable prodrug of gabapentin and a colonically absorbable prodrug of pregabalin. In certain embodiments, a colonically absorbable prodrug of a GABA analog having antispastic activity that is not directly mediated by the GABA_{B} receptor is a colonically absorbable prodrug of gabapentin. In certain embodiments, a colonically absorbable prodrug of a GABA analog having antispastic activity that is not directly mediated by the GABA_{B} receptor is a colonically absorbable prodrug of pregabalin.

In certain embodiments, a colonically absorbable prodrug of gabapentin is chosen from a compound of Formula (I):

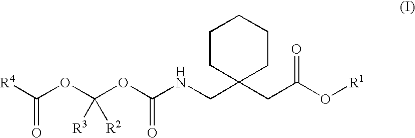

pharmaceutically acceptable salts thereof, pharmaceutically acceptable solvates of any of the foregoing, and pharmaceutically acceptable N-oxides of any of the foregoing, wherein:

R¹ is chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, heterocycloalkyl, substituted heterocycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, heterocycloalkyl, and substituted heterocycloalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl.

In certain embodiments, a colonically absorbable prodrug of pregabalin is chosen from a compound of Formula (II):

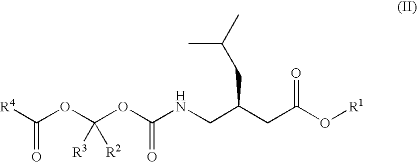

pharmaceutically acceptable salts thereof, pharmaceutically acceptable solvates of any of the foregoing, and pharmaceutically acceptable N-oxides of any of the foregoing, wherein:

R¹ is chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, heterocycloalkyl, substituted heterocycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, heterocycloalkyl, and substituted heterocycloalkyl ring; and

R⁴ is chosen from acyl, substituted acyl, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl.

In certain embodiments of compounds of Formula (I) and Formula (II), each substitute group is independently chosen from halogen, —OH, CN, —CF₃, —C(O)NH₂, —COOR¹⁰, and —NR¹⁰₂ wherein each R¹⁰ is independently chosen from hydrogen and C₁₋₃ alkyl.

In certain embodiments of compounds of Formula (I) and Formula (II), R¹ is hydrogen.

In certain embodiments of compounds of Formula (I) and Formula (II), R² and R³ are independently chosen from hydrogen and C₁₋₆ alkyl.

In certain embodiments of compounds of Formula (I) and Formula (II), one of R² and R³ is C₁₋₆ alkyl and the other of R² and R³ is hydrogen.

In certain embodiments of compounds of Formula (I) and Formula (II), R³ is chosen from methyl, ethyl, n-propyl, isopropyl, n-butyl, isobutyl, and sec-butyl; and R² is hydrogen.

In certain embodiments of compounds of Formula (I) and Formula (II), R³ is chosen from methyl, ethyl, n-propyl, and isopropyl, and R² is hydrogen.

In certain embodiments of compounds of Formula (I) and Formula (II), R⁴ is chosen from C₁₋₆ alkyl and C₁₋₆ substituted alkyl. In certain embodiments of compounds of Formula (I) and Formula (II) wherein R⁴ is chosen from C₁₋₆ substituted alkyl, the substitute group is independently chosen from halogen, —NH₂, OH, —CN, —CF₃, —COOH, —C(O)NH₂, —C(O)OR¹⁰, and —NR^{1O}₂ wherein each R¹⁰ is independently C₁₋₃ alkyl.

In certain embodiments of compounds of Formula (I) and Formula (II), R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formula (I) and Formula (II), R⁴ is chosen from methyl, ethyl, n-propyl, isopropyl, n-butyl, and isobutyl.

In certain embodiments of compounds of Formula (I) and Formula (II), each of R¹ and R² is hydrogen; R³ is C₁₋₆ alkyl; and R⁴ is chosen from C₁₋₆ alkyl and substituted C₁₋₆ alkyl. In certain embodiments of compounds of Formula (I) and Formula (II), each of R¹ and R² is hydrogen; R³ is C₁₋₆ alkyl; and R⁴ is chosen from C₁₋₆ alkyl and substituted C₁₋₆ alkyl, each substitute group is independently chosen from halogen, —NH₂, —OH, —CN, —CF₃, —COOH, —C(O)NH₂, —C(O)OR¹⁰, and —NR¹⁰₂ wherein each R¹⁰ is independently C₁₋₃ alkyl.

In certain embodiments of compounds of Formula (I) and Formula (II), each of R¹ and R² is hydrogen; R³ is chosen from methyl, ethyl, n-propyl, isopropyl, n-butyl, isobutyl, and sec-butyl; and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formula (I) and Formula (II), each of R¹ and R² is hydrogen; R³ is chosen from methyl, ethyl, n-propyl, and isopropyl; and R⁴ is chosen from methyl, ethyl, n-propyl, isopropyl, n-butyl, and isobutyl.

In certain embodiments of the compound of Formula (I) wherein R⁴ is isopropyl, R² is hydrogen, and R³ is methyl; the compound of Formula (I) is 1-{[(α-isobutanoyloxyethoxy)carbonyl]aminomethyl}-1-cyclohexane acetic acid, a pharmaceutically acceptable salt thereof, a pharmaceutically acceptable solvate of any of the foregoing, or a pharmaceutically acceptable N-oxide of any of the foregoing.

In certain embodiments of the compound of Formula (I) wherein R⁴ is isopropyl, R² is hydrogen, and R³ is methyl; the compound of Formula (I) is a crystalline form of 1-{[(α-isobutanoyloxyethoxy)carbonyl]aminomethyl}-1-cyclohexane acetic acid as disclosed in Estrada et al., U.S. Application Publication No. 2005/015405, which is incorporated by reference herein in its entirety. In certain embodiments, crystalline 1-{[(α-isobutanoyloxyethoxy)carbonyl]aminomethyl}-1-cyclohexane acetic acid has characteristic absorption peaks at 7.0°±0.3°, 8.2°±0.3°, 10.5°±0.3°, 12.8°±0.3°, 14.9°±0.3°, 16.4°±0.3°, 17.9°±0.3°, 18.1°±0.3°, 18.9°±0.3°, 20.9°±0.3°, 23.3°±0.3°, 25.3°±0.3°, and 26.6°±0.3° in an X-ray powder diffractogram. In certain embodiments, crystalline 1-{[(α-isobutanoyloxyethoxy)carbonyl]aminomethyl}-1-cyclohexane acetic acid has a melting point range from about 63° C. to about 64° C., in certain embodiments, from about 64° C. to about 66° C., and in certain embodiments, from about 63° C. to about 66° C.

Examples of compounds of Formula (I) include
1-{[(α-acetoxyethoxy)carbonyl]aminomethyl}-1-cyclohexane acetic acid;
1-{[(α-propanoyloxyethoxy)carbonyl]aminomethyl}-1-cyclohexane acetic acid;
1-{[(α-butanoyloxyethoxy)carbonyl]aminomethyl}-1-cyclohexane acetic acid;
1-{[(α-isobutanoyloxyethoxy)carbonyl]aminomethyl}-1-cyclohexane acetic acid;
1-{[(α-pivaloxyethoxy)carbonyl]aminomethyl}-1-cyclohexane acetic acid;
1-{[(α-acetoxymethoxy)carbonyl]aminomethyl}-1-cyclohexane acetic acid;
1-{[(α-propanoyloxymethoxy)carbonyl]aminomethyl}-1-cyclohexane acetic acid;
1-{[(α-butanoyloxymethoxy)carbonyl]aminomethyl}-1-cyclohexane acetic acid;
1-{[(α-isobutanoyloxymethoxy)carbonyl]aminomethyl}-1-cyclohexane acetic acid;
1-{[(α-pivaloxymethoxy)carbonyl]aminomethyl}-1-cyclohexane acetic acid;
1-{[(α-acetoxypropoxy)carbonyl]aminomethyl}-1-cyclohexane acetic acid;
1-{[(α-propanoyloxypropoxy)carbonyl]aminomethyl}-1-cyclohexane acetic acid;
1-{[(α-butanoyloxypropoxy)carbonyl]aminomethyl}-1-cyclohexane acetic acid;
1-{[(α-isobutanoyloxypropoxy)carbonyl]aminomethyl}-1-cyclohexane acetic acid;
1-{[(α-pivaloxypropoxy)carbonyl]aminomethyl}-1-cyclohexane acetic acid;
1-{[(α-acetoxyisopropoxy)carbonyl]aminomethyl}-1-cyclohexane acetic acid;
1-{[(α-propanoyloxyisopropoxy)carbonyl]aminomethyl}-1-cyclohexane acetic acid;
1-{[(α-butanoyloxyisopropoxy)carbonyl]aminomethyl}-1-cyclohexane acetic acid;
1-{[(α-isobutanoyloxyisopropoxy)carbonyl]aminomethyl}-1-cyclohexane acetic acid;
1-{[(α-pivaloxyisopropoxy)carbonyl]aminomethyl}-1-cyclohexane acetic acid;
1-{[(α-acetoxybutoxy)carbonyl]aminomethyl}-1-cyclohexane acetic acid;
1-{[(α-propanoyloxybutoxy)carbonyl]aminomethyl}-1-cyclohexane acetic acid;
1-{[(α-butanoyloxybutoxy)carbonyl]aminomethyl}-1-cyclohexane acetic acid;
1-{[(α-isobutanoyloxybutoxy)carbonyl]aminomethyl}-1-cyclohexane acetic acid;
1-{[(α-pivaloxybutoxy)carbonyl]aminomethyl}-1-cyclohexane acetic acid, and

pharmaceutically acceptable salts of any of the foregoing, pharmaceutically acceptable solvates of any of the foregoing, and pharmaceutically acceptable N-oxides of any of the foregoing.

Examples of compounds of Formula (II) include:
3-{[(α-acetoxyethoxy)carbonyl]aminomethyl}(3S)-5-methyl hexanoic acid;
3-{[(α-propanoyloxyethoxy)carbonyl]aminomethyl}(3S)-5-methyl hexanoic acid;
3-{[(α-butanoyloxyethoxy)carbonyl]aminomethyl}(3S)-5-methyl hexanoic acid;
3-{[(α-isobutanoyloxyethoxy)carbonyl]aminomethyl}(3S)-5-methyl hexanoic acid;
3-{[(α-pivaloxyethoxy)carbonyl]aminomethyl}(3S)-5-methyl hexanoic acid;
3-{[(α-acetoxymethoxy)carbonyl]aminomethyl}(3S)-5-methyl hexanoic acid;
3-{[(α-propanoyloxymethoxy)carbonyl]aminomethyl}(3S)-5-methyl hexanoic acid;
3-{[(α-butanoyloxymethoxy)carbonyl]aminomethyl}(3S)-5-methyl hexanoic acid;
3-{[(α-isobutanoyloxymethoxy)carbonyl]aminomethyl}(3S)-5-methyl hexanoic acid;
3-{[(α-pivaloxymethoxy)carbonyl]aminomethyl}(3S)-5-methyl hexanoic acid;
3-{[(α-acetoxypropoxy)carbonyl]aminomethyl}(3S)-5-methyl hexanoic acid;
3-{[(α-propanoyloxypropoxy)carbonyl]aminomethyl}(3S)-5-methyl hexanoic acid;
3-{[(α-butanoyloxypropoxy)carbonyl]aminomethyl}(3S)-5-methyl hexanoic acid;
3-{[(α-isobutanoyloxypropoxy)carbonyl]aminomethyl}(3S)-5-methyl hexanoic acid;
3-{[(α-pivaloxypropoxy)carbonyl]aminomethyl}(3S)-5-methyl hexanoic acid;
3-{[(α-acetoxyisopropoxy)carbonyl]aminomethyl}(3S)-5-methyl hexanoic acid;
3-{[(α-propanoyloxyisopropoxy)carbonyl]aminomethyl}(3S)-5-methyl hexanoic acid;
3-{[(α-butanoyloxyisopropoxy)carbonyl]aminomethyl}(3S)-5-methyl hexanoic acid;
3-{[(α-isobutanoyloxyisopropoxy)carbonyl]aminomethyl}(3S)-5-methyl hexanoic acid;
3-{[(α-pivaloxyisopropoxy)carbonyl]aminomethyl}(3S)-5-methyl hexanoic acid;
3-{[(α-acetoxybutoxy)carbonyl]aminomethyl}(3S)-5-methyl hexanoic acid;
3-{[(α-propanoyloxybutoxy)carbonyl]aminomethyl}(3S)-5-methyl hexanoic acid;
3-{[(α-butanoyloxybutoxy)carbonyl]aminomethyl}(3S)-5-methyl hexanoic acid;
3-{[(α-isobutanoyloxybutoxy)carbonyl]aminomethyl}(3S)-5-methyl hexanoic acid;

3 {[(α-pivaloxybutoxy)carbonyl]aminomethyl}(3S)-5-methyl hexanoic acid; and

pharmaceutically acceptable salts of any of the foregoing, pharmaceutically acceptable solvates of any of the foregoing, and pharmaceutically acceptable N-oxides of any of the foregoing.

In certain embodiments, a compound of Formula (II) is 3-{[(α-isobutanoyloxyethoxy)carbonyl]aminomethyl}(3S)-5-methyl hexanoic acid, a pharmaceutically acceptable salt thereof, a pharmaceutically acceptable solvate of any of the foregoing, or a pharmaceutically acceptable N-oxide of any of the foregoing.

Methods of synthesizing colonically absorbable prodrugs of GABA analogs, including methods of synthesizing compounds of Formula (I) and (II) are disclosed in Gallop et al., U.S. Pat. Nos. 6,818,787 and 6,972,341; Gallop et al., PCT International Publication No. WO 02/100347; Gallop et al., U.S. Application Publication Nos. 2004/0077553, 2003/0176398, 2003/0171303, and 2004/006132; Raillard et al., U.S. Application Publication No. 2004/0014940; and Bhat et al., U.S. Application Publication No. 2005/0070715, each of which is incorporated by reference herein in its entirety. Other methods of synthesizing prodrugs of GABA analogs have also been disclosed (see e.g., Bryans et al., PCT International Publication No. WO 01/90052; U.K. Application GB 2,362,646; European Applications EP 1,201,240 and 1,178,034; Yatvin et al., U.S. Pat. No. 6,024,977; Gallop et al., PCT International Publication No. WO 02/28881; Gallop et al., PCT International Publication No. WO 02/28883; Gallop et al., International Publication No. WO 02/28411; Gallop et al., PCT International Publication No. WO 02/32376; and Gallop et al., PCT International Publication No. WO 02/42414).

### Antispasticity Agents

Antispasticity agents are compounds shown to be useful in treating spasticity. The biochemical mechanism by which an antispasticity agent exerts its effect is not intended to limit the scope of antispasticity agent. In certain embodiments, an antispasticity agent is chosen from baclofen, R-baclofen, diazepam, tizanidine, clonidine, dantrolene, 4-aminopyridine, cyclobenzaprine, ketazolam, tiagabine, botulinum A toxin, and a prodrug of any of the foregoing. Compounds having activity as an α2δ subunit calcium channel modulator are believed to be useful as antispasticity agents. α2δ-Ligands are described in Dooley et al., U.S. Pat. No. 7,164,034, and U.S. Application Publication Nos. 2004/0186177 and 2007/0027212; and Artman et al., U.S. Pat. No. 6,589,994 and U.S. Application Publication No. 2004/0072900.

### GABA_{B} Receptor Agonists

In certain embodiments, an antispasticity agent is a GABA_{B} receptor agonist. Many examples of compounds having agonistic or partially agonistic activity to GABA_{B} receptors are known and include certain amino acids, aminophosphonic acids, aminophosphinic acids, aminophosphonous acids, and aminosulfinic acids such as, for example:
4-amino-3-(2-chlorophenyl)butanoic acid;
4-amino-3-(4-fluorophenyl)butanoic acid;
4-amino-3-hydroxybutanoic acid;
4-amino-3-(4-chlorophenyl)-3-hydroxyphenylbutanoic acid;
4-amino-3-(thien-2-yl)butanoic acid;
4-amino-3-(5-chlorothien-2-yl)butanoic acid;
4-amino-3-(5-bromothien-2-yl)butanoic acid;
4-amino-3-(5-methylthien-2-yl)butanoic acid;
4-amino-3-(2-imidazolyl)butanoic acid;
4-guanidino-3-(4-chlorophenyl)butanoic acid;
(3-aminopropyl)phosphonous acid;
(4-aminobut-2-yl)phosphonous acid;
(3-amino-2-methylpropyl)phosphonous acid;
(3-aminobutyl)phosphonous acid;
(3-amino-2-(4-chlorophenyl)propyl)phosphonous acid;
(3-amino-2-(4-chlorophenyl)-2-hydroxypropyl)phosphonous acid;
(3-amino-2-(4-fluorophenyl)propyl)phosphonous acid;
(3-amino-2-phenylpropyl)phosphonous acid;
(3-amino-2-hydroxypropyl)phosphonous acid;
(E)-(3-aminopropen-1-yl)phosphonous acid;
(3-amino-2-cyclohexylpropyl)phosphonous acid;
(3-amino-2-benzylpropyl)phosphonous acid;
[3-amino-2-(4-trifluoromethylphenyl)propyl]phosphonous acid;
[3-amino-2-(4-methoxyphenyl)propyl]phosphonous acid;
[3-amino-2-(4-chlorophenyl)-2-hydroxypropyl]phosphonous acid;
(3-aminopropyl)methylphosphinic acid;
(3-amino-2-hydroxypropyl)methylphosphinic acid;
(3-aminopropyl)(difluoromethyl)phosphinic acid;
(4-aminobut-2-yl)methylphosphinic acid;
(3-amino-1-hydroxypropyl)methylphosphinic acid;
(3-amino-2-hydroxypropyl)(difluoromethyl)phosphinic acid;
(E)-(3-aminopropen-1-yl)methylphosphinic acid;
(3-amino-2-oxo-propyl)methyl phosphinic acid;
(3-aminopropyl)hydroxymethylphosphinic acid;
(5-aminopent-3-yl)methylphosphinic acid;
(4-amino-1,1,1-trifluorobut-2-yl)methylphosphinic acid;
3-aminopropylsulfinic acid;
(3-amino-2-(4-chlorophenyl)propyl)sulfinic acid;
(3-amino-2-hydroxypropyl)sulfinic acid;
(2S)-(3-amino-2-hydroxypropyl)sulfinic acid;
(2R)-(3-amino-2-hydroxypropyl)sulfinic acid;
(3-amino-2-fluoropropyl)sulfinic acid;
(2S)-(3-amino-2-fluoropropyl)sulfinic acid;
(2R)-(3-amino-2-fluoropropyl)sulfinic acid;
(3-amino-2-oxopropyl)sulfinic acid;
4-aminobutanoic acid (GABA);
3-(aminopropyl)methylphosphinic acid;
4-amino-3-phenylbutanoic acid;
4-amino-3-hydroxybutanoic acid;
4-amino-3-(4-chlorophenyl)-3-hydroxyphenylbutanoic acid;
4-amino-3-(thien-2-yl)butanoic acid;
4-amino-3-(5-chlorothien-2-yl)butanoic acid;
4-amino-3-(5-bromothien-2-yl)butanoic acid;
4-amino-3-(5-methylthien-2-yl)butanoic acid;
4-amino-3-(2-imidazolyl)butanoic acid;
4-guanidino-3-(4-chlorophenyl)butanoic acid;
3-amino-2-(4-chlorophenyl)-1-nitropropane;
(3-aminopropyl)phosphonous acid;
(4-aminobut-2-yl)phosphonous acid;
(3-amino-2-methylpropyl)phosphonous acid;
(3-aminobutyl)phosphonous acid;
(3-amino-2-(4-chlorophenyl)propyl)phosphonous acid;
(3-amino-2-(4-chlorophenyl)-2-hydroxypropyl)phosphonous acid;
(3-amino-2-(4-fluorophenyl)propyl)phosphonous acid;
(3-amino-2-phenylpropyl)phosphonous acid;
(3-amino-2-hydroxypropyl)phosphonous acid;
(E)-(3-aminopropen-1-yl)phosphonous acid;
(3-amino-2-cyclohexylpropyl)phosphonous acid;
(3-amino-2-benzylpropyl)phosphonous acid;
[3-amino-2-(4-methylphenyl)propyl]phosphonous acid;
[3-amino-2-(4-trifluoromethylphenyl)propyl]phosphonous acid;
[3-amino-2-(4-methoxyphenyl)propyl]phosphonous acid;
[3-amino-2-(4-chlorophenyl)-2-hydroxypropyl]phosphonous acid;
(3-amino propyl)methylphosphinic acid;
(3-amino-2-hydroxypropyl)methylphosphinic acid;
(3-aminopropyl)(difluoromethyl)phosphinic acid;
(4-aminobut-2-yl)methylphosphinic acid;
(3-amino-1-hydroxypropyl)methylphosphinic acid;
(3-amino-2-hydroxypropyl)(difluoromethyl)phosphinic acid;
(E)-(3-aminopropen-1-yl)methylphosphinic acid;
(3-amino-2-oxo-propyl)methylphosphinic acid;
(3-aminopropyl)hydroxymethylphosphinic acid;
(5-aminopent-3-yl)methylphosphinic acid;
(4-amino-1,1,1-trifluorobut-2-yl)methylphosphinic acid;
(3-amino-2-(4-chlorophenyl)propyl)sulfinic acid;
3-aminopropylsulfinic acid; and
1-(aminomethyl)cyclohexaneacetic acid.

Other GABA_{B} receptor agonists include:
3-aminopropylphosphinic acid;
3-aminopropyl-(P-methyl)-phosphinic acid;
β-phenyl-GABA;
baclofen;
3-hydroxy-baclofen;
4-amino-5-methoxybenzofuran-2-yl)-butanoic acid;
4-amino-β-(5-chloro-thien-2-yl)-butanoic acid;
2-aminoethanesulfonic acid;
4-(3-hydroxy-pyridin-2-yl)-butyrolactam, γ-hydroxyburtyrate;
4′-ethyl-2-methyl-3-pyrrolidinopropiophenone;
1-(4-chlorophenyl)-4-(3,5-dimethoxybenzoyl)-piperazine;
4-{[α-(4-chlorophenyl)-5-5-fluoro-2-hydroxybenzylidene]amino}butyramide; and
2-(7-chloro-1,8-naphthyridin-2-yl)-3-[(1,4-dioxa-8-azaspiro[4,5]dec-8-yl)carbonylmethyl]-isoindolin-1-one (see e.g., Kerr and Ong, Pharmac. Ther. 1995, 67(2), 187-246).

Compounds having GABA_{B} receptor agonist activity are also disclosed in Andrews and Lehmann, U.S. Pat. No. 6,664,069; Kaufman and Tian, U.S. Pat. No. 6,350,769; Kaplan et al., U.S. Pat. No. 4,094,992 (progabide: 4-[[4-chlorophenyl)-(5-fluoro-2-hydroxyphenyl)methylene]amino]butamide); Gallop et al., U.S. Pat. No. 7,109,239; Meythaler and Peduzzi, U.S. Application Publication No. 2006/0142396; Kitzpatrick et al., U.S. Application Publication No. 2004/0152775; Lehmann et al., U.S. Application Publication Nos. 2006/0172979 and 2007/0021393; and Elebring et al., U.S. Application Publication Nos. 2002/0156053, 2003/0220303, and 2005/0137414.

In certain embodiments, a GABA_{B} receptor agonist is baclofen.

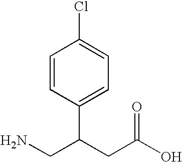

The GABA_{B} receptor agonist, (±)-4-amino-3-(4-chlorophenyl)butanoic acid (baclofen), is an analog of gamma-aminobutyric acid (i.e., GABA) that selectively activates GABA_{B} receptors, resulting in neuronal hyperpolarization. GABA_{B} receptors are located in laminae I-IV of the spinal cord, where primary sensory fibers end. These G-protein coupled receptors activate conductance by K⁺-selective ion channels and can reduce currents mediated by Ca²⁺ channels in certain neurons. Baclofen has a presynaptic inhibitory effect on the release of excitatory neurotransmitters and also acts postsynaptically to decrease motor neuron firing (see Bowery, Trends Pharmacol. Sci. 1989, 10, 401-407; and Misgeld et al., Prog. Neurobiol. 1995, 46, 423-462). A principal pharmacological effect of baclofen in mammals is reduction of muscle tone and the drug is frequently used in the treatment of spasticity.

Baclofen may be administered orally or by intrathecal delivery through a surgically implanted programmable pump. The drug is rapidly absorbed from the gastrointestinal tract and exhibits an elimination half-life of approximately 3-4 hours. Baclofen is partially metabolized in the liver but is largely excreted by the kidneys unchanged. The short half-life of baclofen necessitates frequent administration with typical oral dosing regimens ranging from about 10 to about 80 mg of three or four divided doses daily. Plasma baclofen concentrations of about 80 to about 400 ng/mL result from these therapeutically effective doses in patients (Katz, Am. J. Phys. Med. Rehabil. 1988, 2, 108-116; and Krach, J. Child Neurol. 2001, 16, 31-36). When baclofen is given orally, sedation is a side effect, particularly at elevated doses. Impairment of cognitive function, confusion, memory loss, dizziness, weakness, ataxia, and orthostatic hypotension are other commonly encountered baclofen side-effects.

Intrathecal administration is often recommended for patients who find the adverse effects of oral baclofen intolerable. The intrathecal use of baclofen permits effective treatment of spasticity with doses less than 1/100^{th} of those required orally, since administration directly into the spinal subarachnoid space permits immediate access to the GABA_{B} receptor sites in the dorsal horn of the spinal cord. Surgical implantation of a pump is, however, inconvenient and a variety of mechanical and medical complications can anse, e.g., catheter displacement, kinking or blockage, pump failure, sepsis and deep vein thrombosis. Acute discontinuation of baclofen therapy, such as caused by mechanical failure, may cause serious withdrawal symptoms such as hallucinations, confusion, agitation and seizures (Sampathkumar et al., Anesth. Analg. 1998, 87, 562-563).

While the clinically prescribed baclofen product (Lioresal™) is available only as a racemate, the GABA_{B} receptor agonist activity resides entirely in the R-enantiomer, R-(−)-baclofen (4) (also termed L-baclofen).

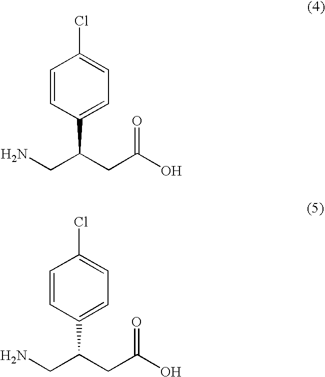

The other isomer, S-baclofen (5), antagonizes the action of R-baclofen at GABA_{B} receptors and its antinociceptive activity in the rat spinal cord (Terrence et al., Pharmacology 1983, 27, 85-94; and Sawynok et al., Pharmacology 1985, 31, 248-259). Orally administered R-baclofen is reported to be about 5-fold more potent than orally administered racemic baclofen, with an R-baclofen regimen of 2 mg t.i.d being equivalent to racemic baclofen at 10 mg t.i.d. (Fromm et al., Neurology 1987, 37, 1725-1728). Moreover, the side effect profile, following administration of R-baclofen, has been shown to be significantly reduced, relative to an equally efficacious dose of racemic baclofen.

### Colonically Absorbable GABA_{B} Receptor Agonists

Baclofen, a zwitterionic amino acid, lacks the requisite physicochemical characteristics for effective passive permeability across cellular membranes. Passage of the drug across the gastrointestinal tract and the blood-brain barrier (BBB) is mediated primarily by active transport processes rather than by passive diffusion. Accordingly, baclofen is a substrate for active transport mechanisms shared by neutral α-amino acids such as leucine, and β-amino acids such as β-alanine and taurine (van Bree et al., Pharm. Res. 1988, 5, 369-371; Cercos-Fortea et al., Biopharm. Drug. Disp. 1995, 16, 563-577; Deguchi et al., Pharm. Res. 1995, 12, 1838-1844; and Moll-Navarro et al., J. Pharm. Sci. 1996, 85, 1248-1254). Transport across the BBB is stereoselective, with preferential uptake of the active R-enantiomer (4) being reported (van Bree et al., Pharm. Res. 1991, 8, 259-262). In addition, organic anion transporters localized in capillary endothelial cells of the blood-brain barrier have been implicated in efflux of baclofen from the brain (Deguchi et al., Pharm Res 1995, 12, 1838-44; and Ohtsuki et al., J. Neurochem. 2002, 83, 57-66). 3-(p-Chlorophenyl)pyrrolidine has been described as a CNS-penetrable prodrug of baclofen (Wall et al., J. Med. Chem. 1989, 32, 1340-1348). Colonically absorbable prodrugs of GABA_{B} receptor agonists are described in Gallop et al., U.S. Pat. Nos. 7,109,239 and 6,972,341, and U.S. Application Publication Nos. 2003/0176398 and 2005/022243, each of which is incorporated by reference herein in its entirety.

In certain embodiments, a colonically absorbable prodrug of a GABA_{B} receptor agonist is a colonically absorbable prodrug of R-baclofen.

In certain embodiments, a colonically absorbable prodrug of a GABA_{B} receptor agonist is chosen from a compound of Formula (III):

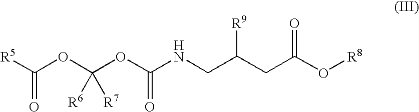

pharmaceutically acceptable salts thereof, pharmaceutically acceptable solvates of any of the foregoing, and pharmaceutically acceptable N-oxides of any of the foregoing, wherein:

R⁵ is chosen from acyl, substituted acyl, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;

R⁶ and R⁷ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R⁶ and R⁷ together with the carbon atom to which they are bonded form a ring chosen from a cycloalkyl, substituted cycloalkyl, heterocycloalkyl, and substituted heterocycloalkyl ring;

R⁸ is chosen from hydrogen, alkyl, substituted alkyl, aryl, substituted aryl, arylalkyl, substituted arylalkyl, aryldialkylsilyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, and trialkylsilyl; and

R⁹ is chosen from substituted aryl, heteroaryl, and substituted heteroaryl.

In certain embodiments of a compound of Formula (III), R⁸ is hydrogen.

In certain embodiments of a compound of Formula (III), R⁶ and R⁷ are chosen from hydrogen and C₁₋₆ alkyl.

In certain embodiments of a compound of Formula (III), one of R⁶ and R⁷ is C₁₋₆ alkyl and the other of R⁶ and R⁷ is hydrogen.

In certain embodiments of a compound of Formula (III), R⁵ is chosen from C₁₋₆ alkyl and substituted C₁₋₆ alkyl.

In certain embodiments of a compound of Formula (III), R⁵ is C₁₋₆ alkyl; R⁶ is C₁₋₆ alkyl, R⁷ is hydrogen; and R⁸ is hydrogen.

In certain embodiments of a compound of Formula (III), R⁵ is chosen from methyl, ethyl, n-propyl, isopropyl, n-butyl, isobutyl, sec-butyl, tert-butyl, n-pentyl, isopentyl, sec-pentyl, neopentyl, 1,1-diethoxyethyl, phenyl, cyclohexyl, 2-pyridyl, 3-pyridyl, and 4-pyridyl; R⁶ is chosen from hydrogen, methyl, ethyl, n-propyl, isopropyl, n-butyl, isobutyl, sec-butyl, phenyl, and cyclohexyl; R⁷ is hydrogen; and R⁸ is hydrogen.

In certain embodiments of a compound of Formula (III), R⁵ is methyl, ethyl, n-propyl, isopropyl, n-butyl, isobutyl, sec-butyl, tert-butyl, phenyl, cyclohexyl, and 3-pyridyl; R⁶ is hydrogen; R⁷ is hydrogen; and R⁸ is hydrogen.

In certain embodiments of a compound of Formula (III), R⁵ is chosen from methyl, ethyl, n-propyl, isopropyl, n-butyl, isobutyl, sec-butyl, tert-butyl, phenyl, and cyclohexyl; R⁶ is chosen from methyl, n-propyl, and isopropyl; R⁷ is hydrogen; and R⁸ is hydrogen.

In certain embodiments of a compound of Formula (III), R⁵ is chosen from methyl, ethyl, n-propyl, isopropyl, n-butyl, isobutyl, sec-butyl, tert-butyl, phenyl, and cyclohexyl; R⁶ is isopropyl; R⁷ is hydrogen; and R⁸ is hydrogen.

In certain embodiments of a compound of Formula (III), R⁵ is isopropyl; R⁶ is isopropyl; R⁷ is hydrogen; and R⁸ is hydrogen.

In certain embodiments of a compound of Formula (III), R⁹ is chosen from 4-chlorophenyl, (R)-4-chlorophenyl, 2-chlorophenyl, 4-fluorophenyl, thien-2-yl, 5-chlorothien-2-yl, 5-bromothien-2-yl, 5-methylthien-2-yl, and 2-imidazolyl.

In certain embodiments of a compound of Formula (III), R⁹ is 4-chlorophenyl and the carbon to which R⁹ is bonded is of the R-configuration; i.e. (R)-4-chlorophenyl.

In certain embodiments of a compound of Formula (III), each substitute group is independently chosen from halogen, —OH, —CN, —CF₃, —C(O)NH₂, —COOR¹⁰, and —NR¹⁰₂ wherein each R¹⁰ is independently chosen from hydrogen and C₁₋₃ alkyl.

In certain embodiments of a compound of Formula (III), the compound is a colonically absorbable prodrug of R-baclofen of Formula (IV):

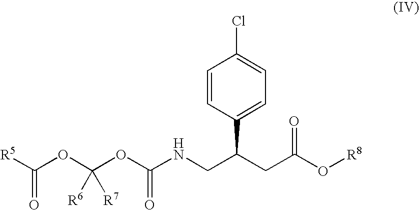

wherein R⁵, R⁶, R⁷, and R⁸ are as defined as for compounds of Formula (III).

In certain embodiments of a compound of Formula (IV), the compound is (3R)-4-{[(1S)-2-methyl-1-(2-methylpropanoyloxy)propoxy]carbonylamino}-3-(4-chlorophenyl)butanoic acid.

Methods of synthesizing colonically absorbable prodrugs of R-baclofen are disclosed, for example, in Gallop et al., U.S. Pat. Nos. 6,933,140, 7,109,239, 7,186,855, 7,227,028, and 7,300,956; U.S. Application Publication Nos. 2004/0198820, and 2007/0010453, each of which is incorporated by reference herein in its entirety.

### Methods of Use

Colonically absorbable prodrugs of GABA analogs having antispastic activity that is not directly mediated by the GABA_{B} receptor, optionally in combination with an antispasticity agent or a colonically absorbable prodrug of a GABA_{B} receptor agonist, may be administered to a patient for treating spasticity.

Spasticity is estimated to affect about 500,000 people in the United States and more than 12 million people worldwide. Spasticity is an involuntary, velocity-dependent, increased resistance to stretch. Spasticity is characterized by muscle hypertonia and displays increased resistance to externally imposed movement with increasing speed of stretch (Lance et al., Trans Am. Neurol. Assoc. 1970, 95, 272-274; and Sanger et al., Pediatrics 2003, 111, e89-e97). Spasticity can be caused by lack of oxygen to the brain before, during, or after birth (cerebral palsy); physical trauma (brain or spinal cord injury); blockage of or bleeding from a blood vessel in the brain (stroke); certain metabolic diseases; adrenolekodystrophy; phenylketonuria; neurodegenerative diseases such as Parkinson's disease and amyotrophic lateral sclerosis; and neurological disorders such as multiple sclerosis. Spasticity is associated with damage to the corticospinal tract and is a common complication of neurological disease. Diseases and conditions in which spasticity may be a prominent symptom include cerebral palsy, multiple sclerosis, stroke, head and spinal cord injuries, traumatic brain injury, anoxia, and neurodegenerative diseases. Patients with spasticity complain of stiffness, involuntary spasm, and pain. These painful spasms may be spontaneous or triggered by a minor sensory stimulus, such as touching the patient.

Symptoms of spasticity can include hypertonia (increased muscle tone), clonus (a series of rapid muscle contractions), exaggerated deep tendon reflexes, muscle spasms, scissoring (involuntary crossing of the legs), deformities with fixed joints, stiffness, and/or fatigue caused by trying to force the limbs to move normally. Other complications include urinary tract infections, chronic constipation, fever or other systemic illnesses, and/or pressure sores. The degree of spasticity varies from mild muscle stiffness to severe, painful, and uncontrollable muscle spasms. Spasticity may coexist with other conditions but is distinguished from rigidity (involuntary bidirectional non-velocity-dependent resistance to movement), clonus (self-sustaining oscillating movements secondary to hypertonicity), dystonia (involuntary sustained contractions resulting in twisting abnormal postures), athetoid movement (involuntary irregular confluent writhing movements), chorea (involuntary, abrupt, rapid, irregular, and unsustained movements), ballisms (involuntary flinging movements of the limbs or body), and tremor (involuntary rhythmic repetitive oscillations, not self-sustaining). Spasticity can lead to orthopedic deformity such as hip dislocation, contractures, or scoliosis; impairment of daily living activities such as dressing, bathing, and toileting; impairment of mobility such as inability to walk, roll, or sit; skin breakdown secondary to positioning difficulties and shearing pressure; pain or abnormal sensory feedback; poor weight gain secondary to high caloric expenditure; sleep disturbance; and/or depression secondary to lack of functional independence.

Spasticity can be assessed using methods and procedures known in the art such as a combination of clinical examination; the use of rating scales such as the Ashworth Scale, the modified Ashworth Scale, the Spasm Frequency Scale, and the Reflex Score; biomechanical studies such as the pendulum test; electrophysiologic studies including electromyography; and functional measurements such as the Fugl-Meyer Assessment of Sensorimotor Impairment scale. Other spasticity scales have been developed to assess spasticity of a specific etiology such as the Multiple Sclerosis Spasticity Scale (MSS-88) (Hobart et al., Brain 2006, 129(1), 224-234).

Treatment of spasticity includes physical and occupational therapy such as functional based therapies, rehabilitation, facilitation such as neurodevelopmental therapy, proprioceptive neuromuscular facilitation, and sensory integration; biofeedback; electrical stimulation; and orthoses. Oral medications useful in treating spasticity include baclofen, benzodiazepines such as diazepam, dantrolene sodium; imidazolines such as clonidine and tizanidine; and gabapentin. Intrathecal medications useful in treating spasticity include baclofen. Chemodenervation with local anesthetics such as lidocaine and xylocaine; type A botulinum toxin and type B botulinum toxin; phenol and alcohol injection can also be useful in treating spasticity. Surgical treatments useful in treating spasticity include neurosurgery such as selective dorsal rhizotomy; and orthopedic operations such as contracture release, tendon or muscle lengthening, tendon transfer, osteotomy, and arthrodesis.

In certain embodiments, a colonically absorbable prodrug of a GABA analog having antispastic activity that is not directly mediated by the GABA_{B} receptor or pharmaceutical composition thereof may be administered to a patient suffering from spasticity. The suitability of a colonically absorbable prodrug of a GABA analog or pharmaceutical compositions thereof to treat spasticity may be determined by methods known to those skilled in the art.

When used in the present methods of treatment, upon releasing a colonically absorbable prodrug of a GABA analog in vivo, a dosage form comprising a prodrug of a colonically absorbable prodrug of a GABA analog having antispastic activity that is not directly mediated by the GABA_{B} receptor or pharmaceutical composition thereof provides the corresponding GABA analog (e.g., in certain embodiments, gabapentin or pregabalin) in the systemic circulation of a patient. The promoiety or promoieties of the prodrug may be cleaved either chemically and/or enzymatically. One or more enzymes present in the intestinal lumen, intestinal tissue, blood, liver, brain, or any other suitable tissue of a mammal may cleave the promoiety or promoieties of the prodrug. The mechanism of cleavage is not important to the current methods. In certain embodiments, a GABA analog that is formed by cleavage of the promoiety or promoieties from the corresponding GABA analog prodrug does not contain substantial quantities of lactam contaminant (such as, less than about 0.5% by weight, for example, less than about 0.2% by weight, and in certain embodiments, less than about 0.1% by weight) for the reasons described in Augart et al., U.S. Pat. No. 6,054,482. The extent of release of lactam contaminant from a GABA analog prodrug may be assessed using standard in vitro analytical methods.

Colonically absorbable prodrugs of GABA analogs having antispastic activity that is not directly mediated by the GABA_{B} receptor, for example the gabapentin prodrug 1-{[(α-isobutanoyloxyethoxy)carbonyl]aminomethyl}-1-cyclohexane acetic acid may be more efficacious than the parent drug molecule (e.g., gabapentin or other GABA analog) in treating spasticity because colonically absorbable prodrugs of GABA analogs when taken orally and facilitate the ability to maintain plasma concentrations within a therapeutically effective window for a prolonged period of time. It is believed that colonically absorbable prodrugs of GABA analogs, for example, the gabapentin prodrug 1-{[(α-isobutanoyloxyethoxy)carbonyl]aminomethyl}-1-cyclohexane acetic acid, are absorbed from the gastrointestinal lumen into the blood by a different mechanism than that by which gabapentin and other known GABA analogs are absorbed. For example, gabapentin is believed to be actively transported across the gut wall by a carrier transporter localized in the human small intestine. The gabapentin transporter is easily saturated which means that the amount of gabapentin absorbed into the blood may not be proportional to the amount of gabapentin that is administered orally, because once the transporter is saturated, further absorption of gabapentin does not occur to any significant degree. In comparison to gabapentin, colonically absorbable prodrugs of GABA analogs, for example, the gabapentin prodrug 1-{[(α-isobutanoyloxyethoxy)carbonyl]aminomethyl}-1-cyclohexane acetic acid, are believed to be absorbed across the gut wall along a greater portion of the gastrointestinal tract, including the colon. Because colonically absorbable prodrugs of GABA analogs can be effectively formulated in sustained release formulations, which provide for sustained release of a GABA analog prodrug into the gastrointestinal tract, for example, within the colon, over a period of hours, the compounds, such as the gabapentin prodrug 1-{[α-isobutanoyloxyethoxy)carbonyl]aminomethyl}-1-cyclohexane acetic acid, may be more efficacious than their respective parent drugs (e.g., gabapentin or other GABA analog) in treating spasticity. The ability of colonically absorbable prodrugs of GABA analogs to be used in sustained release oral dosage forms may reduce the dosing frequency necessary for maintenance of a therapeutically effective drug concentration in the systemic circulation.

Dosage forms comprising a colonically absorbable prodrug of a GABA analog having antispastic activity that is not directly mediated by the GABA_{B} receptor may be administered or applied singly or in combination with another colonically absorbable prodrug of a GABA analog or with other pharmacological agents. Dosage forms may also deliver a colonically absorbable prodrug of a GABA analog to a patient in combination with another pharmacologically active agent including another colonically absorbable prodrug of a GABA analog and/or another active agent known or believed to be capable of treating spasticity.

In certain embodiments, colonically absorbable prodrugs of GABA analogs having antispastic activity that is not directly mediated by the GABA_{B} receptor are suitable for oral administration, wherein the promoiety or promoieties are cleaved after absorption of the GABA analog prodrug by the gastrointestinal tract (e.g., in intestinal tissue, blood, liver or other suitable tissue of the patient) following oral administration of the corresponding colonically absorbable GABA analog prodrug. The promoiety or promoieties may render the prodrug a substrate for one or more transporters expressed in the large intestine (i.e., colon), and/or, for GABA analogs that are poorly absorbed across the gastrointestinal mucosa (e.g., gabapentin and pregabalin), may facilitate the ability of the prodrug to be passively absorbed across the gastrointestinal mucosa.

### Colonically Absorbable GABA Analog Prodrugs and Antispasticity Agents

In certain embodiments, a colonically absorbable prodrug of a GABA analog having antispastic activity that is not directly mediated by the GABA_{B} receptor in combination with an antispasticity agent, or pharmaceutical composition thereof, may be administered to a patient suffering from spasticity. The suitability of a colonically absorbable prodrug of a GABA analog and antispasticity agent, or pharmaceutical compositions thereof, to treat spasticity may be determined by methods known to those skilled in the art.

In certain embodiments, a colonically absorbable prodrug of a GABA analog having antispastic activity that is not directly mediated by the GABA_{B} receptor in combination with an antispasticity agent chosen from baclofen, R-baclofen, diazepam, tizanidine, clonidien, dantrolene, 4-aminopyridine, cyclobenzaprine, ketazolam, tiagabine, botulinum A toxin, and a prodrug of any of the foregoing, or pharmaceutical composition thereof, may be administered to a patient for treating spasticity. In certain embodiments, a colonically absorbable prodrug of a GABA analog in combination with R-baclofen, or pharmaceutical composition thereof, may be administered to a patient for treating spasticity.

In certain embodiments, a colonically absorbable prodrug of gabapentin and/or a colonically absorbable prodrug of pregabalin in combination with an antispasticity agent, or pharmaceutical composition thereof, may be administered to a patient for treating spasticity. In certain embodiments, a colonically absorbable prodrug of gabapentin and/or a colonically absorbable prodrug of pregabalin in combination with an antispasticity agent chosen from baclofen, R-baclofen, diazepam, tizanidine, clonidien, dantrolene, 4-aminopyridine, cyclobenzaprine, ketazolam, tiagabine, botulinum A toxin, and a prodrug of any of the foregoing, or pharmaceutical composition thereof, may be administered to a patient for treating spasticity. In certain embodiments, a colonically absorbable prodrug of gabapentin and/or a colonically absorbable prodrug of pregabalin in combination with R-baclofen, or pharmaceutical composition thereof, may be administered to a patient for treating spasticity.

In certain embodiments, a compound of Formula (I) and/or Formula (II) in combination with an antispasticity agent, or pharmaceutical composition thereof, may be administered to a patient for treating spasticity. In certain embodiments, a compound of Formula (I) and/or Formula (II) in combination with an antispasticity agent chosen from baclofen, R-baclofen, diazepam, tizanidine, clonidine, dantrolene, 4-aminopyridine, cyclobenzaprine, ketazolam, tiagabine, botulinum A toxin, and a prodrug of any of the foregoing, or pharmaceutical composition thereof, may be administered to a patient for treating spasticity. In certain embodiments, a compound of Formula (I) and/or Formula (II) in combination with R-baclofen, or pharmaceutical composition thereof, may be administered to a patient for treating spasticity.

In certain embodiments, 1-{[α-isobutanoyloxyethoxy)carbonyl]aminomethyl}-1-cyclohexane acetic acid and/or 3-{[α-isobutanoyloxyethoxy)carbonyl]aminomethyl}(3S)-5-methyl hexanoic acid in combination with an antispasticity agent, or pharmaceutical composition thereof, may be administered to a patient suffering from spasticity. In certain embodiments, 1-{[(α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with an antispasticity agent chosen from baclofen, R-baclofen, diazepam, tizanidine, clonidine, dantrolene, 4-aminopyridine, cyclobenzaprine, ketazolam, tiagabine, botulinum A toxin, and a prodrug of any of the foregoing, or pharmaceutical composition thereof, may be administered to a patient for treating spasticity. In certain embodiments, 1-{[(α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with R-baclofen, or pharmaceutical composition thereof, may be administered to a patient for treating spasticity.

### Colonically Absorbable Prodrugs of a GABA Analog and Colonically Absorbable Prodrugs of a GABA_{B} Receptor Agonist

In certain embodiments, a colonically absorbable prodrug of a GABA analog having antispastic activity that is not directly mediated by the GABA_{B} receptor in combination with a colonically absorbable prodrug of a GABA_{B} receptor agonist, or pharmaceutical composition thereof, may be administered to a patient suffering from spasticity. The suitability of a prodrug of a GABA analog and prodrug of a GABA_{B} receptor agonist, or pharmaceutical compositions thereof to treat spasticity may be determined by methods known to those skilled in the art.

In certain embodiments, a colonically absorbable prodrug of gabapentin and/or a colonically absorbable prodrug of pregabalin in combination with a colonically absorbable prodrug of a GABA_{B} receptor agonist, or pharmaceutical composition thereof, may be administered to a patient for treating spasticity. In certain embodiments, a compound of Formula (I) and/or Formula (II) in combination with a colonically absorbable prodrug of a GABA_{B} receptor agonist, or pharmaceutical composition thereof, may be administered to a patient for treating spasticity. In certain embodiments, 1-{[(α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with a colonically absorbable prodrug of a GABA_{B} receptor agonist, or pharmaceutical composition thereof, may be administered to a patient for treating spasticity.

In certain embodiments, a colonically absorbable prodrug of a GABA analog having antispastic activity that is not directly mediated by the GABA_{B} receptor in combination with a colonically absorbable prodrug of R-baclofen, or pharmaceutical composition thereof, may be administered to a patient suffering from spasticity. In certain embodiments, a colonically absorbable prodrug of gabapentin and/or a colonically absorbable prodrug of pregabalin in combination with a colonically absorbable prodrug of R-baclofen, or pharmaceutical composition thereof, may be administered to a patient for treating spasticity. In certain embodiments, a compound of Formula (I) and/or Formula (II) in combination with a colonically absorbable prodrug of R-baclofen, or pharmaceutical composition thereof, may be administered to a patient for treating spasticity. In certain embodiments, 1-{[(α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with a colonically absorbable prodrug of R-baclofen, or pharmaceutical composition thereof, may be administered to a patient for treating spasticity.

In certain embodiments, a colonically absorbable prodrug of a GABA analog having antispastic activity that is not directly mediated by the GABA_{B} receptor in combination with a compound of Formula (III), or pharmaceutical composition thereof, may be administered to a patient suffering from spasticity. In certain embodiments, a colonically absorbable prodrug of gabapentin and/or a colonically absorbable prodrug of pregabalin in combination with a compound of Formula (III), or pharmaceutical composition thereof, may be administered to a patient for treating spasticity. In certain embodiments, a compound of Formula (I) and/or Formula (II) in combination with a compound of Formula (III), or pharmaceutical composition thereof, may be administered to a patient for treating spasticity.

In certain embodiments, 1-{[α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with a compound of Formula (III), or pharmaceutical composition thereof, may be administered to a patient for treating spasticity.

In certain embodiments, 1-{[(α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with a compound of Formula (IV), or pharmaceutical composition thereof, may be administered to a patient for treating spasticity.

In certain embodiments, a colonically absorbable prodrug of a GABA analog having antispastic activity that is not directly mediated by the GABA_{B} receptor in combination with (3R)-4-{[(1S)-2-methyl-1-(2-methylpropanoyloxy)propoxy]carbonylamino}-3-(4-chlorophenyl)butanoic acid, or pharmaceutical composition thereof, may be administered to a patient suffering from spasticity. In certain embodiments, a colonically absorbable prodrug of gabapentin and/or a colonically absorbable prodrug of pregabalin in combination with (3R)-4-{[(1S)-2-methyl-1-(2-methylpropanoyloxy)propoxy]carbonylamino}-3-(4-chlorophenyl)butanoic acid, or pharmaceutical composition thereof, may be administered to a patient for treating spasticity. In certain embodiments, a compound of Formula (I) and/or Formula (II) in combination with (3R)-4-{[(1S)-2-methyl-1-(2-methylpropanoyloxy)propoxy]carbonylamino}-3-(4-chlorophenyl)butanoic acid, or pharmaceutical composition thereof, may be administered to a patient for treating spasticity.

In certain embodiments, 1-{[(α-isobutanoyloxyethoxy)carbonyl]aminomethyl}-1-cyclohexane acetic acid and/or 3-{[(α-isobutanoyloxyethoxy)carbonyl]aminomethyl}(3S)-5-methyl hexanoic acid in combination with (3R)-4-{[(1S)-2-methyl-1-(2-methylpropanoyloxy)propoxy]carbonylamino}-3-(4-chlorophenyl)butanoic acid, or pharmaceutical composition thereof, may be administered to a patient for treating spasticity.

In certain embodiments, a method of treating spasticity in a patient comprises administering to a patient in need of such treatment a colonically absorbable prodrug of gabapentin of Formula (I):

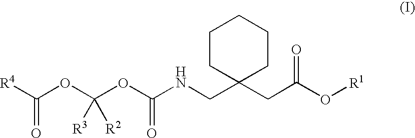

or a pharmaceutically acceptable salt thereof, wherein each of R¹ and R² is hydrogen; R³ is C₁₋₆ alkyl; and R⁴ is chosen from C₁₋₆ alkyl and substituted C₁₋₆ alkyl; and a colonically absorbable prodrug of a GABA_{B} agonist of Formula (IV):

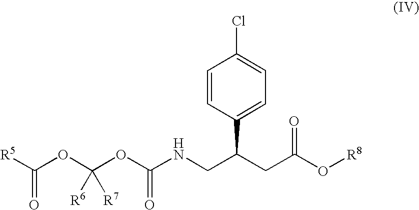

or a pharmaceutically acceptable salt thereof, wherein each of R⁷ and R⁸ is hydrogen; R⁶ is C₁₋₆ alkyl; and R⁵ is chosen from C₁₋₆ alkyl and substituted C₁₋₆ alkyl.

In certain embodiments, a method of treating spasticity in a patient comprises administering to a patient in need of such treatment a colonically absorbable prodrug of pregabalin of Formula (II):

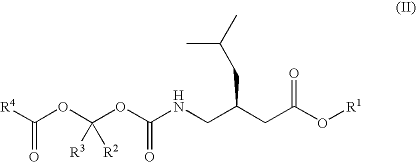

or a pharmaceutically acceptable salt thereof, wherein each of R¹ and R² is hydrogen; R³ is C₁₋₆ alkyl; and R⁴ is chosen from C₁₋₆ alkyl and substituted C₁₋₆ alkyl; and a colonically absorbable prodrug of a GABA_{B} agonist of Formula (IV):

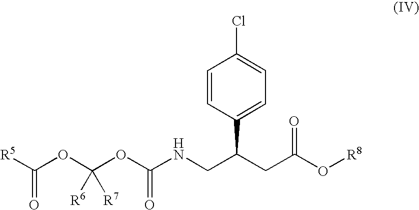

or a pharmaceutically acceptable salt thereof, wherein each of R⁷ and R⁸ is hydrogen; R⁶ is C₁₋₆ alkyl; and R⁵ is chosen from C₁₋₆ alkyl and substituted C₁₋₆ alkyl.

### Pharmaceutical Compositions

Colonically absorbable GABA analog prodrugs having antispastic activity that is not directly mediated by the GABA_{B} receptor; colonically absorbable GABA analog prodrugs having antispastic activity that is not directly mediated by the GABA_{B} receptor and antispasticity agents; and colonically absorbable GABA analog prodrugs and colonically absorbable prodrugs of GABA_{B} receptor agonists may be provided as pharmaceutical compositions. Pharmaceutical compositions provided by the present disclosure comprise at least one colonically absorbable GABA analog prodrug and at least one pharmaceutically acceptable vehicle; at least one colonically absorbable GABA analog prodrugs and at least one antispasticity agent and at least one pharmaceutically acceptable vehicle; or at least one colonically absorbable GABA analog prodrug and at least one colonically absorbable prodrug of a GABA_{B} receptor agonist, and at least one pharmaceutically acceptable vehicle. A pharmaceutical composition may comprise a therapeutically effective amount of at least one colonically absorbable GABA analog prodrug; a therapeutically effective amount of at least one colonically absorbable GABA analog prodrugs and at least one antispasticity agent; or a therapeutically effective amount of at least one colonically absorbable GABA analog prodrug and at least one colonically absorbable prodrug of a GABA_{B} receptor agonist; either individually or in combination; and at least one pharmaceutically acceptable vehicle. A therapeutically effective amount refers to the amount of each compound individually, or both compounds together. In certain embodiments, a pharmaceutical composition may comprise more than one colonically absorbable GABA analog prodrug, more than one antispasticity agent, and/or more than one colonically absorbable prodrug of a GABA_{B} receptor agonist. Pharmaceutically acceptable vehicles include diluents, adjuvants, excipients, and carriers.

Pharmaceutical compositions may be produced using standard procedures (see e.g., “Remington's The Science and Practice of Pharmacy,” 21st edition, Lippincott, Williams & Wilcox, 2005). Pharmaceutical compositions may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of compounds disclosed herein into preparations, which can be used pharmaceutically. Proper formulation can depend, in part, on the route of administration.

Pharmaceutical compositions provided by the present disclosure may provide therapeutic levels of a colonically absorbable prodrug of a GABA analog having antispastic activity that is not directly mediated by the GABA_{B} receptor, antispasticity agent, and/or colonically absorbable prodrug of a colonically absorbable prodrug of a GABA_{B} receptor agonist upon administration to a patient. The promoiety or promoieties of a GABA analog prodrug or GABA_{B} receptor agonist prodrug may be cleaved in vivo either chemically and/or enzymatically to release the corresponding GABA analog or GABA_{B} receptor agonist. In certain embodiments, a GABA analog prodrug or GABA_{B} receptor agonist prodrug is essentially not metabolized to release the corresponding GABA analog or GABA_{B} receptor agonist within enterocytes, but is metabolized to the parent drug within the systemic circulation. Cleavage of the promoiety or promoieties of a GABA analog prodrug or GABA_{B} receptor agonist after absorption by the gastrointestinal tract may allow the corresponding prodrug to be absorbed into the systemic circulation either by active transport, passive diffusion, or by a combination of both active and passive processes.

GABA analog prodrugs having antispastic activity that is not directly mediated by the GABA_{B} receptor and GABA_{B} receptor agonist prodrugs may remain intact until after passage of the prodrug through a biological barrier, such as the blood-brain barrier. In certain embodiments, prodrugs provided by the present disclosure may be partially cleaved, e.g., one or more, but not all, of the promoieties can be cleaved before passage through a biological barrier or prior to being taken up by a cell, tissue, or organ. GABA analog prodrugs and GABA_{B} receptor agonist prodrugs may remain intact in the systemic circulation and be absorbed by cells of an organ, either passively or by active transport mechanisms. In certain embodiments, a GABA analog prodrug or GABA_{B} receptor agonist prodrug will be lipophilic and can passively translocate through cellular membranes. Following cellular uptake, the GABA analog prodrug or GABA_{B} receptor agonist prodrugs may be cleaved chemically and/or enzymatically to release the corresponding GABA analog or corresponding GABA_{B} receptor agonist into the cellular cytoplasm, resulting in an increase in the intracellular concentration of the GABA analog or GABA_{B} receptor agonist.

In certain embodiments, a pharmaceutical composition may include an adjuvant that facilitates absorption of a colonically absorbable prodrug of a GABA analog having antispastic activity that is not directly mediated by the GABA_{B} receptor r, an antispasticity agent, and/or a GABA_{B} receptor agonist prodrug through the gastrointestinal epithelia. Such enhancers may, for example, open the tight-junctions in the gastrointestinal tract or modify the effect of cellular components, such as p-glycoprotein and the like. Suitable enhancers can include alkali metal salts of salicylic acid, such as sodium salicylate, caprylic or capric acid, such as sodium caprylate or sodium caprate, and the like. Enhancers can include, for example, bile salts, such as sodium deoxycholate. Various p-glycoprotein modulators are described in Fukazawa et al., U.S. Pat. No. 5,112,817 and Pfister et al., U.S. Pat. No. 5,643,909. Various absorption enhancing compounds and materials are described in Burnside et al., U.S. Pat. No. 5,824,638, and Meezam et al., U.S. Application Publication No. 2006/0046962. Other adjuvants that enhance permeability of cellular membranes include resorcinol, surfactants, polyethylene glycol, and bile acids.

In certain embodiments, a pharmaceutical composition may include an adjuvant that reduces enzymatic degradation of a prodrug of a GABA analog having antispastic activity that is not directly mediated by the GABA_{B} receptor, an antispasticity agent, and/or a GABA_{B} receptor agonist prodrug. Microencapsulation using protenoid microspheres, liposomes, or polysaccharides can also be effective in reducing enzymatic degradation of administered compounds

A pharmaceutical composition may also include one or more pharmaceutically acceptable vehicles, including excipients, adjuvants, carriers, diluents, binders, lubricants, disintegrants, colorants, stabilizers, surfactants, fillers, buffers, thickeners, emulsifiers, wetting agents, and the like. Vehicles may be selected to alter the porosity and permeability of a pharmaceutical composition, alter hydration and disintegration properties, control hydration, enhance manufacturability, etc.

In certain embodiments, a pharmaceutical composition may be formulated for oral administration. Pharmaceutical compositions formulated for oral administration may provide for uptake of a prodrug of a GABA analog having antispastic activity that is not directly mediated by the GABA_{B} receptor, an antispasticity agent, and/or a GABA_{B} receptor agonist prodrug throughout the gastrointestinal tract, or in a particular region or regions of the gastrointestinal tract. In certain embodiments, a pharmaceutical composition may be formulated to enhance uptake of a GABA analog prodrug, an antispasticity agent, and/or a GABA_{B} receptor agonist prodrug from the lower gastrointestinal tract, and in certain embodiments, from the large intestine, including the colon. Such compositions may be prepared in a manner known in the pharmaceutical art and may further comprise, in addition to a GABA analog prodrug, an antispasticity agent, and/or a GABA_{B} receptor agonist prodrug, one or more pharmaceutically acceptable vehicles, permeability enhancers, and/or a second therapeutic agent.

In certain embodiments, a pharmaceutical composition may further comprise substances to enhance, modulate and/or control release, bioavailability, therapeutic efficacy, therapeutic potency, stability, and the like. For example, to enhance therapeutic efficacy, a prodrug of a GABA analog having antispastic activity that is not directly mediated by the GABA_{B} receptor, an antispasticity agent, and/or a GABA_{B} receptor agonist prodrug may be co-administered with one or more active agents to increase the absorption or diffusion of at least one compound of a GABA analog prodrug, an antispasticity agent, and/or a GABA_{B} receptor agonist prodrug from the gastrointestinal tract, or to inhibit degradation of the drug in the systemic circulation. In certain embodiments, a GABA analog prodrug, an antispasticity agent, and/or a GABA_{B} receptor agonist prodrug may be co-administered with active agents having pharmacological effects that enhance the therapeutic efficacy of a GABA analog prodrug, an antispasticity agent, and/or a GABA_{B} receptor agonist prodrug.

Pharmaceutical compositions may take the form of solutions, suspensions, emulsions, tablets, pills, pellets, capsules, capsules containing liquids, powders, sustained-release formulations, suppositories, emulsions, aerosols, sprays, mists, suspensions, or any other appropriate form suitable for use.

Pharmaceutical compositions comprising a prodrug of a GABA analog having antispastic activity that is not directly mediated by the GABA_{B} receptor, an antispasticity agent, and/or a GABA_{B} receptor agonist prodrug may be formulated for oral administration. Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame and/or saccharin, flavoring agents such as peppermint, oil of wintergreen, cherry, or other suitable flavorings, coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions may include standard vehicles such as mannitol, lactose, starch, magnesium stearate, sodium saccharine, cellulose, magnesium carbonate, etc. Such vehicles may be of pharmaceutical grade.

When a prodrug of a GABA analog having antispastic activity that is not directly mediated by the GABA_{B} receptor, an antispasticity agent, and/or a GABA_{B} receptor agonist prodrug is acidic, it may be provided as the free acid, a pharmaceutically acceptable salt, a solvate, or a hydrate. Pharmaceutically acceptable salts substantially retain the activity of the free acid, may be prepared by reaction with bases, and tend to be more soluble in aqueous and other protic solvents than the corresponding free acid form. In some embodiments, salts of a GABA analog prodrug, an antispasticity agent, and/or a GABA_{B} receptor agonist prodrug may be used in a formulation. In certain embodiments, the salt can be an alkali metal salt such as hydrogen or sodium, and in certain embodiments the salt can be an alkali earth metal salt such as calcium.

Pharmaceutical compositions provided by the present disclosure may be formulated so as to provide immediate, sustained, or delayed release of a compound of a prodrug of a GABA analog having antispastic activity that is not directly mediated by the GABA_{B} receptor, an antispasticity agent, and/or a GABA_{B} receptor agonist prodrug after administration to a patient by employing procedures known in the art (see, e.g., Allen et al., “Ansel's Pharmaceutical Dosage Forms and Drug Delivery Systems,” 8th edition, Lippincott, Williams & Wilkins, August 2004). In certain embodiments, a pharmaceutical composition comprising a GABA analog prodrug, an antispasticity agent, and/or a GABA_{B} receptor agonist prodrug may be formulated for sustained release formulation.

### Dosage Forms

Pharmaceutical compositions provided by the present disclosure may be formulated in a unit dosage form. A unit dosage form refers to a physically discrete unit suitable as a unitary dose for patients undergoing treatment, with each unit containing a predetermined quantity of a prodrug of a GABA analog having antispastic activity that is not directly mediated by the GABA_{B} receptor, an antispasticity agent, and/or a GABA_{B} receptor agonist prodrug calculated to produce the intended therapeutic effect. A unit dosage form may be for a single daily dose, 1 to 2 times per day, or one of multiple daily doses, e.g., 2 to 4 times per day. When multiple daily doses are used, a unit dosage may be the same or different for each dose. One or more dosage forms may comprise a dose, which may be administered to a patient at a single point in time or during a time interval.

Pharmaceutical compositions provided by the present disclosure may be used in dosage forms that provide immediate release and/or controlled release of at least one compound of a prodrug of a GABA analog having antispastic activity that is not directly mediated by the GABA_{B} receptor, an antispasticity agent, and/or a GABA_{B} receptor agonist prodrug. The appropriate type of dosage form can depend on the etiology and/or severity of the spasticity being treated, and on the method of administration. In certain embodiments, dosage forms may be adapted to be administered to a patient no more than twice per day, and in certain embodiments, only once per day. Dosing may be provided alone or in combination with other drugs and may continue as long as required for effective treatment of the disease, disorder, or condition.

Pharmaceutical compositions comprising a prodrug of a GABA analog having antispastic activity that is not directly mediated by the GABA_{B} receptor, an antispasticity agent, and/or a GABA_{B} receptor agonist prodrug may be formulated for immediate release for oral administration, or by any other appropriate route of administration.

In certain embodiments, oral dosage forms provided by the present disclosure may be controlled release dosage forms. Controlled delivery technologies can improve the absorption of a drug in a particular region or regions of the gastrointestinal tract. Controlled drug delivery systems may be designed to deliver a drug in such a way that the drug level is maintained within a therapeutically effective blood concentration range and effective and safe blood levels are maintained for a period as long as the system continues to deliver the drug at a particular rate. Controlled drug delivery may produce substantially constant blood levels of a drug as compared to fluctuations observed with immediate release dosage forms administered by the same route of administration. For some drugs, maintaining a constant blood and tissue concentration throughout the course of therapy is the most desirable mode of treatment. Immediate release of these drugs may cause blood levels to peak above the level required to elicit the desired response, which wastes the drug and may cause or exacerbate toxic side effects. Controlled drug delivery may result in optimum therapy, and not only may reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

The appropriate oral dosage form for a particular pharmaceutical composition provided by the present disclosure can depend, at least in part, on the gastrointestinal absorption properties of the compound of a prodrug of a GABA analog having antispastic activity that is not directly mediated by the GABA_{B} receptor, an antispasticity agent, and/or a GABA_{B} receptor agonist prodrug, the stability of a GABA analog prodrug, an antispasticity agent, and/or a GABA_{B} receptor agonist prodrug in the gastrointestinal tract, the pharmacokinetics of the compound of a GABA analog prodrug, an antispasticity agent, and/or a GABA_{B} receptor agonist prodrug, and an intended therapeutic profile. An appropriate controlled release oral dosage form may be selected for a particular compound of a GABA analog prodrug, an antispasticity agent, and/or a GABA_{B} receptor agonist prodrug. For example, gastric retention oral dosage forms may be appropriate for compounds absorbed primarily from the upper gastrointestinal tract, and sustained release oral dosage forms may be appropriate for compounds absorbed primarily from the lower gastrointestinal tract.

Pharmaceutical compositions provided by the present disclosure may be practiced with a number of different dosage forms, which can be adapted to provide sustained release of at least one compound of a prodrug of a GABA analog having antispastic activity that is not directly mediated by the GABA_{B} receptor, an antispasticity agent, and/or a GABA_{B} receptor agonist prodrug upon oral administration. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's Pharmaceutical Sciences,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

Sustained release oral dosage forms may be in any appropriate form for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, and the like.

In certain embodiments, sustained release oral dosage forms may comprise a therapeutically effective amount of a colonically absorbable prodrug of a GABA analog having antispastic activity that is not directly mediated by the GABA_{B} receptor, an antispasticity agent, and/or a colonically absorbable prodrug of a GABA_{B} receptor agonist and a pharmaceutically acceptable vehicle. In certain embodiments, sustained release oral dosage forms may comprise less than a therapeutically effective amount of a GABA analog prodrug, an antispasticity agent, and/or a GABA_{B} receptor agonist prodrug and a pharmaceutically effective vehicle. Multiple sustained release oral dosage forms, each dosage form comprising less than a therapeutically effective amount of a GABA analog prodrug, an antispasticity agent, and/or a GABA_{B} receptor agonist prodrug may be administered at a single time or over a period of time to provide a therapeutically effective dose or regimen for treating spasticity.

Sustained release oral dosage forms provided by the present disclosure may release a colonically absorbable prodrug of GABA analog having antispastic activity that is not directly mediated by the GABA_{B} receptor, an antispasticity agent, and/or a colonically absorbable prodrug of a GABA_{B} receptor agonist from the dosage form to facilitate the ability of a GABA analog prodrug, an antispasticity agent, and/or a GABA_{B} receptor agonist prodrug to be absorbed from an appropriate region of the gastrointestinal tract, for example, in the colon. In certain embodiments, sustained release oral dosage forms release a GABA analog prodrug, an antispasticity agent, and/or a GABA_{B} agonist prodrug from the dosage form over a period of at least about 4 hours, at least about 8 hours, at least about 12 hours, at least about 16 hours, at least about 20 hours, and in certain embodiments, at least about 24 hours. In certain embodiments, sustained release oral dosage forms release a GABA analog prodrug, an antispasticity agent, and/or a GABA_{B} receptor agonist prodrug from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 24 hours. In certain embodiments, sustained release oral dosage forms release a GABA analog prodrug, an antispasticity agent, and/or a GABA_{B} receptor agonist prodrug from the dosage form in a delivery pattern of from about 0 wt % to about 20 wt % in about 0 to about 4 hours, about 20 wt % to about 50 wt % in about 0 to about 8 hours, about 55 wt % to about 85 wt % in about 0 to about 14 hours, and about 80 wt % to about 100 wt % in about 0 to about 20 hours. In certain embodiments, sustained release oral dosage forms release a GABA analog prodrug, an antispasticity agent, and/or a GABA_{B} agonist prodrug from the dosage form in a delivery pattern of from about 0 wt % to about 20 wt % in about 0 to about 2 hours, about 20 wt % to about 50 wt % in about 0 to about 4 hours, about 55 wt % to about 85 wt % in about 0 to about 7 hours, and about 80 wt % to about 100 wt % in about 0 to about 8 hours.

Sustained release oral dosage forms comprising at least one colonically absorbable prodrug of a GABA analog having antispastic activity that is not directly mediated by the GABA_{B} receptor, antispasticity agent, and/or colonically absorbable prodrug of a GABA_{B} receptor agonist may provide a concentration of the corresponding GABA analog, antispasticity agent, and/or corresponding GABA_{B} receptor agonist in the plasma, blood, or tissue of a patient over time, following oral administration to the patient. The concentration profile of a GABA analog, antispasticity agent, and/or GABA_{B} receptor agonist may exhibit an AUC that is proportional to the dose of the corresponding GABA analog prodrug, antispasticity agent, and/or corresponding GABA_{B} receptor agonist prodrug.

Regardless of the specific type of controlled release oral dosage form used, a colonically absorbable prodrug of a GABA analog having antispastic activity that is not directly mediated by the GABA_{B} receptor, an antispasticity agent, and/or a colonically absorbable prodrug of a GABA_{B} receptor agonist may be released from an orally administered dosage form over a sufficient period of time to provide prolonged therapeutic concentrations of a GABA analog, an antispasticity agent, and/or a GABA_{B} receptor agonist in the plasma and/or blood of a patient that is effective for treating spasticity. Following oral administration, an oral dosage form comprising a GABA analog prodrug, an antispasticity agent, and/or a GABA_{B} receptor agonist prodrug can provide a therapeutically effective concentration of the corresponding GABA analog, antispasticity agent, and/or GABA_{B} receptor agonist in the plasma and/or blood of a patient for a continuous time period of at least about 4 hours, of at least about 8 hours, for at least about 12 hours, for at least about 16 hours, and in certain embodiments, for at least about 20 hours following oral administration of the dosage form to the patient. The continuous time periods during which a therapeutically effective concentration of a GABA analog, antispasticity agent, and/or GABA_{B} receptor agonist is maintained may be the same or different. The continuous period of time during which a therapeutically effective plasma concentration of a GABA analog, antispasticity agent, and/or GABA_{B} receptor agonist is maintained may begin shortly after oral administration or after a time interval.

In certain embodiments, dosage forms can release from about 0% to about 30% of the a colonically absorbable prodrug of a GABA analog having antispastic activity that is not directly mediated by the GABA_{B} receptor, an antispasticity agent, and/or a colonically absorbable prodrug of a GABA_{B} receptor agonist in from about 0 to about 2 hours, from about 20% to about 50% of the prodrug in from about 2 to about 12 hours, from about 50% to about 85% of the prodrug in from about 3 to about 20 hours and greater than about 75% of the prodrug in from about 5 to about 18 hours. In certain embodiments, sustained release oral dosage forms can provide a concentration profile of a GABA analog, antispasticity agent, and/or GABA_{B} receptor agonist in the blood and/or plasma of a patient over time, which has an area under the curve (AUC) that is proportional to the dose of the corresponding GABA analog prodrug, antispasticity agent, and/or GABA_{B} receptor agonist prodrug administered, and a maximum concentration Cₘₐₓ. In certain embodiments, the Cₘₐₓ may be less than about 75%, and in certain embodiments, may be less than about 60%, of the Cₘₐₓ obtained from administering an equivalent dose of the compound from an immediate release oral dosage form and the AUC is substantially the same as the AUC obtained from administering an equivalent dose of the prodrug from an immediate release oral dosage form.

In certain embodiments, dosage forms may be administered twice per day, and in certain embodiments, once per day, to provide a therapeutically effective concentration of a GABA analog, e.g., gabapentin or pregabalin, antispasticity agent, e.g., baclofen, R-baclofen, diazepam, tizanidine, clonidine, dantrolene, 4-aminopyridine, cyclobenzaprine, ketazolam, tiagabine, botulinum A toxin, or a prodrug of any of the foregoing, and/or a GABA_{B} receptor agonist, e.g., R-baclofen, in the systemic circulation of a patient.

In certain embodiments, oral administration of an oral sustained release dosage form comprising a colonically absorbable prodrug of a GABA analog having antispastic activity that is not directly mediated by the GABA_{B} receptor, antispasticity agent, and/or a colonically absorbable prodrug of a GABA_{B} receptor agonist can provide a therapeutically effective concentration of the corresponding GABA analog, antispasticity agent, and/or GABA_{B} receptor agonist in the blood plasma of a patient for a time period of at least about 4 hours after administration of the dosage form, in certain embodiments, for a time period of at least about 8 hours, and in certain embodiments, for a time period of at least about 12 hours, and in certain embodiments, for a time period of at least about 24 hours.

Examples of sustained release oral dosage forms of colonically absorbable prodrugs of GABA analogs are disclosed, for example in Cundy et al., U.S. Pat. No. 6,833,140, U.S. Application Publication Nos. 2004/0198820 and 2006/0141034, and J Pharm Exptl Ther, 2004, 311, 324-333, and sustained release oral dosage forms comprising colonically absorbable prodrugs of GABA_{B} receptor agonists are disclosed in Kidney et al., U.S. application Ser. No. 11/972,575 filed Jan. 10, 2008; and Sastry et al., U.S. application Ser. No. 12/024,830 filed Feb. 1, 2008, each of which is incorporated by reference herein in its entirety.

In certain embodiments, a colonically absorbable prodrug of a GABA analog having antispastic activity that is not directly mediated by the GABA_{B} receptor, antispasticity agent, and/or colonically absorbable prodrug of a GABA_{B} receptor agonist, or pharmaceutical composition of any of the foregoing may be delivered to a patient via sustained release dosage forms, for example, via oral sustained release dosage forms. When used to treat spasticity a therapeutically effective amount of one or more GABA analog prodrugs of a prodrug of a GABA analog, antispasticity agent, and/or a prodrug of a GABA_{B} receptor agonist may be administered or applied singly or in combination with other agents. A therapeutically effective amount of a prodrug of a GABA analog, antispasticity agent, and/or a prodrug of a GABA_{B} receptor agonist may also deliver the prodrug of a GABA analog, antispasticity agent, and/or prodrug of a GABA_{B} receptor agonist in combination with another pharmaceutically active agent. For example, in the treatment of a patient suffering from spasticity, a dosage form comprising a prodrug of a GABA analog, antispasticity agent, and/or a prodrug of a GABA_{B} receptor agonist may be administered in conjunction with a therapeutic agent known or believed to be capable of treating spasticity, at least one symptom of spasticity, or at least one condition associated with spasticity.

The amount of a colonically absorbable prodrug of a GABA analog having antispastic activity that is not directly mediated by the GABA_{B} receptor, antispasticity agent, and/or a prodrug of a colonically absorbable GABA_{B} agonist that will be effective in treating spasticity will depend, in part, on the nature of the condition and can be determined by standard clinical techniques known in the art. In addition, in vitro or in vivo assays may be employed to help identify optimal dosage ranges. A therapeutically effective amount of a prodrug of a GABA analog, antispasticity agent, and/or a prodrug of a GABA_{B} receptor agonist to be administered may also depend on, among other factors, the subject being treated, the weight of the subject, the severity of the spasticity, the etiology of the spasticity, the manner of administration and the judgment of the prescribing physician.

A therapeutically effective dose may be estimated initially from in vitro assays. Initial doses may also be estimated from in vivo data, e.g., animal models, using techniques that are known in the art. For example, a dose may be formulated in animal models to achieve a beneficial circulating composition concentration range. Such information may be used to more accurately determine useful doses in humans. One having ordinary skill in the art may optimize administration to humans based on animal data.

Suitable dosage ranges for oral administration may depend on the potency of the particular GABA analog, antispasticity agent, or GABA_{B} receptor agonist (once cleaved from the promoiety or promoieties) but may be from about 0.1 mg to about 200 mg of drug per kilogram body weight per day, for example, from about 1 to about 100 mg/kg-body weight per day. In certain embodiments, a compound of Formula (I) may be administered to a patient in an amount from about 10 mg-equivalents to about 3600 mg-equivalents of gabapentin per day, in certain embodiments, from about 200 mg-equivalents to about 2400 mg-equivalents of gabapentin per day, and in certain embodiments, from about 400 mg-equivalents to about 1600 mg-equivalents of gabapentin per day, to treat spasticity. In certain embodiments, a compound of Formula (II) may be administered to a patient in an amount from about 10 mg-equivalents to about 1200 mg-equivalents of pregabalin per day, in certain embodiments, from about 50 mg-equivalents to about 800 mg-equivalents of pregabalin per day, and in certain embodiments, from about 100 mg-equivalents to about 600 mg-equivalents of pregabalin per day to treat spasticity. Dosage ranges may be determined by methods known to those skilled in the art.

In certain embodiments, a compound of Formula (IV) may be administered to a patient in an amount from about 1 mg-equivalents to about 200 mg-equivalents of R-baclofen per day, and in certain embodiments, from about 5 mg-equivalents to about 100 mg-equivalents of R-baclofen per day, to treat spasticity.

A dose may be administered in a single dosage form or in multiple dosage forms. When multiple dosage forms are used the amount of compound contained within each dosage form may be the same or different. The amount of a colonically absorbable prodrug of a GABA analog, antispasticity agent, and/or a colonically absorbable prodrug of a GABA_{B} receptor agonist contained in a dose may depend on the route of administration and whether the spasticity in a patient is effectively treated by acute, chronic, or a combination of acute and chronic administration.

Oral administration comprises orally administering at least one sustained release oral dosage form comprising a colonically absorbable prodrug of a GABA analog having antispastic activity not mediated by the GABA_{B} receptor and an antispasticity agent, and administering at least one first sustained release oral dosage form comprising a colonically absorbable prodrug of a GABA analog having antispastic activity not mediated by the GABA_{B} receptor and at least one second sustained release dosage form comprising a antispasticity agent.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. A dose of a pharmaceutical composition provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Efficacy Assessment

The efficacy of administering a colonically absorbable prodrug of a GABA analog having antispastic activity that is not directly mediated by the GABA_{B} receptor, antispasticity agent, and/or a colonically absorbable prodrug of a GABA_{B} receptor agonist for treating spasticity can be assessed using animal models of spasticity and in clinically relevant studies of spasticity of different etiologies. The therapeutic activity may be determined without determining a specific mechanism of action. Animal models of spasticity are known (See e.g., Eaton, J Rehab Res Dev 2003, 40(4), 41-54; Kakinohana et al., Neuroscience 2006, 141, 1569-1583; Ligresti et al., British J Pharm 2006, 147, 83-91; Zhang et al., Chinese J Clin Rehab, 2006, 10(38), 150-151; Hefferan et al., Neuroscience Letters 2006, 403, 195-200; and Li et al., J Neurophysiol 2004, 92, 2694-2703). For example, animal models of spasticity include (a) the mutant spastic mouse; (b) the acute/chronic spinally transected rat and the acute decerebrate rate; (c) primary observation Irwin Test in the rat; and d) Rotarod Test in the rat and mouse.

The mutant spastic mouse is a homozygous mouse that carries an autosomal recessive trait of genetic spasticity characterized by a deficit of glycine receptors throughout the central nervous system (Chai et al., Proc. Soc. Exptl. Biol. Med. 1962, 109, 491). The mouse is normal at birth and subsequently develops a coarse tremor, abnormal gait, skeletal muscle rigidity, and abnormal righting reflexes at two to three weeks of age. Assessment of spasticity in the mutant spastic mouse can be performed using electrophysiological measurements or by measuring the righting reflex (any righting reflex over one second is considered abnormal), tremor (holding mice by their tails and subjectively rating tremor), and flexibility.

Models of acute spasticity including the acute decerebrate rat, the acute or chronic spinally transected rat, and the chronically spinal cord-lesioned rat (see e.g., Wright and Rang, Clin Orthop Relat Res 1990, 253, 12-19; Shimizu et al., J Pharmacol Sci 2004, 96, 444-449; and Li et al., J Neurophysiol 2004, 92, 2694-2703). The acute models, although valuable in elucidating the mechanisms involved in the development of spasticity, have come under criticism due to the fact that they are acute. The animals usually die or have total recovery from spasticity. The spasticity develops immediately upon intervention, unlike the spasticity that evolves in the human condition of spasticity, which most often initially manifests as a flaccid paralysis. Only after weeks and months does spasticity develop in humans. Some of the more chronic-lesioned or spinally transected models of spasticity do show flaccid paralysis postoperatively. At approximately four weeks post-lesion/transection, the flaccidity changes to spasticity of variable severity. Although all of these models have particular disadvantages and may lack true representation of the human spastic condition, they are shown to be useful in understanding spasticity. These models have also provided methods to test various treatment paradigms that have led to similar treatments being tested in humans. Many of these models also may employ different species, such as cats, dogs, and primates. Baclofen, diazepam, and tizanidine, effective antispasticity agents in humans, are effective on different parameters of electrophysiologic assessment of muscle tone in these models.

The Irwin Test is used to detect physiological, behavioral, and toxic effects of a test substance, and indicates a range of dosages that can be used for later experiments (Irwin, Psychopharmacologia 1968, 13, 222-57). Typically, rats (three per group) are administered a test compound and are then observed in comparison with a control group given vehicle. Behavioral modifications, symptoms of neurotoxicity, pupil diameter, and rectal temperature are recorded according to a standardized observation grid. The grid contains the following items: mortality, sedation, excitation, aggressiveness, Straub tail; writhes, convulsions, tremor, exopthalmos, salivation, lacrimation, piloerection, defecation, fear, traction, reactivity to touch, loss of righting reflexes, sleep, motor incoordination, muscle tone, stereotypes, head-weaving, catalepsy, grasping, ptosis, respiration, corneal reflex, analgesia, abnormal gait, forepaw treading, loss of balance, head twitches, rectal temperature, and pupil diameter. Observations are performed, for example, at 15, 30, 60, 120, and 180 minutes following administration of the test substance, and also 24 hours later. The test substance can be administered intraperitoneally, subcutaneously, or orally.

In the Rotarod Test (Dunham et al., J. Am. Pharm. Assoc. 1957, 46, 208-09) rats or mice are placed on a rod rotating at a speed of eight turns per minute. The number of animals that drop from the rod before three minutes is counted and the drop-off times are recorded (maximum: 180 sec). Diazepam, a benzodiazepine, can be administered at 8 mg/kg intraperitoneally as a reference substance.

Other animal models include spasticity induced in rats following transient spinal cord ischemia (Kakinohana et al., Neuroscience 2006, 141, 1569-1583; and Hefferan et al., Neuroscience Letters 2006, 403, 195-200), spasticity in mouse models of multiple sclerosis (Ligresti et al., British J Pharmacol 2006, 147, 83-91); and spasticity in rat models of cerebral palsy (Zhang et al., Chinese J Clin Rehabilitation 2006, 10(38), 150-151).

The efficacy of colonically absorbable prodrugs of GABA analogs having antispastic activity that is not directly mediated by the GABA_{B} receptor, antispasticity agents, and/or colonically absorbable prodrugs of GABA_{B} receptor agonists in treating spasticity may also be assessed in humans using randomized double-blind placebo-controlled clinical trials (see e.g., Priebe et al., Spinal Cord 1997, 35(3), 171-5; Gruenthal et al., Spinal Cord 1997, 35(10), 686-9; and Tuszynski et al., Spinal Cord 2007, 45, 222-231 and Steeves et al., Spinal Cord 2007, 45, 206-221, for examples of the conduct and assessment of clinical trials for spasticity caused by spinal cord injury). Clinical trial outcome measures for spasticity include the Ashworth Scale, the modified Ashworth Scale, muscle stretch reflexes, presence of clonus and reflex response to noxious stimuli. Other measures can be used to assess spasticity associated with a specific disorder such as the Multiple Sclerosis Spasticity Scale (Hobart et al., Brain 2006, 129(1), 224-234).

### Combination Therapy

In certain embodiments, a prodrug of a colonically absorbable GABA analog having antispastic activity that is not directly mediated by the GABA_{B} receptor, antispasticity agent, and/or a colonically absorbable prodrug of a GABA_{B} receptor agonist, or pharmaceutical compositions of any of the foregoing may be used in combination therapy with at least one other therapeutic agent including a different colonically absorbable prodrug of a GABA analog, antispasticity agent, and/or colonically absorbable prodrug of a GABA_{B} receptor agonist. A colonically absorbable prodrug of a GABA analog, antispasticity agent, and/or a colonically absorbable prodrug of a GABA_{B} receptor agonist, or pharmaceutical composition of any of the foregoing and the additional therapeutic agent may act additively or, in certain embodiments, synergistically, such that the combination of the therapeutic agents together are, for example, more effective, safer, and/or produce fewer or less severe side effects. In certain embodiments, a colonically absorbable prodrug of a GABA analog, antispasticity agent, and/or a colonically absorbable prodrug of a GABA_{B} receptor agonist, or a pharmaceutical composition of any of the foregoing can be administered concurrently with the administration of another therapeutic agent. In certain embodiments, a colonically absorbable prodrug of a GABA analog, antispasticity agent, and/or a colonically absorbable prodrug of a GABA_{B} receptor agonist, or pharmaceutical composition of any of the foregoing can be administered prior or subsequent to administration of another therapeutic agent and thus can have regimens with overlapping schedules. The additional therapeutic agent may be effective for treating spasticity, may be effective in treating at least one symptom of spasticity, may be effective in treating a side effect of administering a colonically absorbable prodrug of a GABA analog, antispasticity agent, and/or a colonically absorbable prodrug of a GABA_{B} receptor agonist for treating spasticity, or may be effective for treating a disease, disorder, or condition other than spasticity. In certain embodiments, in which a colonically absorbable prodrug of a GABA analog, antispasticity agent, and/or a prodrug of a GABA_{B} receptor agonist is administered together with an additional therapeutic agent for treating spasticity each of the active agents may be used at lower doses than when used singly.

In certain embodiments, a colonically absorbable prodrug of a GABA analog having antispastic activity that is not directly mediated by the GABA_{B} receptor, antispasticity agent, and/or a colonically absorbable prodrug of a GABA_{B} receptor agonist may be administered concurrently with the administration of another therapeutic agent, which may be part of the same pharmaceutical composition or dosage form, or in a different composition or dosage form than that containing the compounds provided by the present disclosure. In certain embodiments, a prodrug of a GABA analog, antispasticity agent, and/or a prodrug of a GABA_{B} receptor agonist may be administered prior or subsequent to administration of an additional therapeutic agent. In certain embodiments of combination therapy, the combination therapy comprises alternating between administering a prodrug of a GABA analog, antispasticity agent, and/or a prodrug of a GABA_{B} receptor agonist and a composition comprising an additional therapeutic agent, e.g., to minimize adverse side effects associated with a particular drug. When a prodrug of a GABA analog, antispasticity agent, and/or a prodrug of a GABA_{B} receptor agonist is administered concurrently with another therapeutic agent that potentially can produce adverse side effects including, but not limited to, toxicity, the therapeutic agent may advantageously be administered at a dose that falls below the threshold at which the adverse side effect is elicited.

The weight ratio of a colonically absorbable prodrug of a GABA analog having antispastic activity that is not directly mediated by the GABA_{B} receptor, antispasticity agent, and/or a colonically absorbable prodrug of a GABA_{B} receptor agonist to an additional therapeutic agent may be varied and may depend upon the effective dose of each agent. A therapeutically effective dose of each compound will be used. Thus, for example, when a prodrug of a GABA analog, antispasticity agent, and/or a prodrug of a GABA_{B} receptor agonist is combined with another therapeutic agent, the weight ratio of the prodrug of a GABA analog, antispasticity agent, and/or prodrug of a GABA_{B} receptor agonist to other therapeutic agent can be from about 1000:1 to about 1:1000, and in certain embodiments, from about 200:1 to about 1:200.

### EXAMPLES

The invention is further defined by reference to the following examples, which describe assays for determining GABA_{B} receptor agonist activity, pharmacokinetics of colonically absorbable prodrugs of GABA analogs having antispastic activity that is not directly mediated by the GABA_{B} receptor and colonically absorbable prodrugs of GABA_{B} receptor agonists, and use of prodrugs of GABA analogs having antispastic activity not involving direct action at GABA_{B} receptors, and combinations of prodrugs of GABA analogs having antispastic activity not involving direct action at GABA_{B} receptors and antispasticity agents or GABA_{B} receptor agonists, for treating spasticity.

### Example 1

### Electrophysiology Assay for Determining GABA_{B} Receptor Agonist Activity

GABA_{B} receptor agonist activity was determined using an electrophysiological method employing inward rectification of G-protein-coupled K⁺ channels (GIRK1/4) in Xenopus laevis Oocytes expressing the GABA_{B} receptor (GABA_{B}R 1a/2).

Expression of GABA_{B}R/GIRK in Xenopus laevis Oocytes was accomplished using the following procedure. Oocytes were removed from mature, anesthetized, HCG-injected female Xenopus laevis and washed in 0 mM CaCl₂ ND96 buffer (90 mM NaCl, 10 mM hemi-Na HEPES, 2 mM KCl, 1 mM MgCl₂). Oocytes were then shaken in collagenase solution for 1 h at room temperature. The oocytes were then washed thoroughly and sorted according to desired maturity and morphology. Selected oocytes were injected with a mixture of cRNA encoding for hGBBR1a+2 and rGIRK1+4. Final volume ratios of the GIRK1/4 and GBBR1a/2 RNA were about 1:10 and about 1:5, respectively. Forty-six (46) nL of the RNA mixture was injected per oocyte. Uninjected oocytes were also used as controls. Oocytes were incubated at 16-18° C. in 0.9 mM CaCl₂ ND96 buffer pH 7.4 (90 mM NaCl₂, 10 mM hemi-Na HEPES, 2 mM KCl, 1 mM MgCl₂, 0.9 mM CaCl₂) containing Pen/Strep (SV30010, Hyclone) for 1-2 days.

Electrophysiology measurements were made using a 2-electrode voltage clamp recording instrument (GeneClamp 500B amplifier/Clampex8.2/Clampfit8, Axon Instruments, Union City, Calif.) and standard analysis software (Chart4, ADInstruments, Mountain View, Calif.).

Dose response curves of test compound GABA_{B} agonist activity and pEC50 values were determined as follows. Test compounds were weighed and dissolved in an appropriate solvent. Serial dilution curves were made in 100 mM KCl ND96 buffer (90 mM NaCl₂, 10 mM hemi-Na HEPES, 1 mM MgCl₂, 1.8 mM CaCl₂, 100 mM KCl). The highest concentration of the test compound is usually 1 mM, with 1:5 or 1:4 serial dilutions to provide a 5- or 6-point curve over a concentration range to 0.01 μM. Currents were measured with oocytes clamped at a holding potential between −15 to −40 mV, depending on the health and/or the receptor expression level of individual oocytes. Baseline currents at this holding potential were allowed to reach a steady state before compound addition and recording.

Prior to and between each series of test compound dilutions, a sub-maximal concentration of a known agonist (4 μM GABA) was used as a control. Currents were measured by manually adding 650 μL of diluted test compound to a clamped oocyte in the holding chamber. Currents were allowed to reach saturation before activating the system vacuum/bath perfusion to wash away the test compound. If a test compound appeared to have agonist activity, it was also tested in the presence of a known GABA_{B}R inhibitor (CGP55845). Serial dilutions of the test compound were made in 100 mM KCl ND96 buffer containing 10 μM CGP55845. As another control, the test compound was also tested in uninjected oocytes at a single concentration of 100 μM.

For analysis of the dose response curves, currents generated from each test dilution were calculated as a percentage of the current generated by the control compound. The curve traces were then graphed using GraphPad (Prism, San Diego, Calif.) and pEC50 values generated.

### Example 2

### Ca²⁺ Assay for Determining GABA_{B} Receptor Agonist Activity

The following procedure was used to determine the GABA_{B} receptor agonist activity of a compound as reflected by activation of Ca²⁺ signaling. HEK TREx cells expressing GABA_{B}R1a2 under tetracycline induction control, and Gqi chimeric protein (expressed constitutively), allowing GABA_{B} R coupling to the Ca²⁺ signaling pathway were used in the experiments.

Cells were seeded in media containing tetracycline containing overnight at 100,000 cells/well, in black clear-bottom, 96 well plates. The following morning, cells were washed twice with 100 μL HBSS buffer per well. Fluorescent Ca²⁺ indicator dye is prepared using the materials and procedure described in the F362056 Fluo-4 NW Calcium Assay Kit (Invitrogen, Carlsbad, Calif.). 10 mL of kit buffer and 100 μl of kit Probenecid were added to individual kit dye vials, and rolled back and forth several times to allow dye to dissolve. Cells were then loaded into the dye solution at 50 μL per well. The cells and dye were incubated for 30 min at 37° C., and then incubated for an additional 30 min at room temperature in the dark. Test compounds are dissolved in HBSS buffer at twice final concentration. Duplicate wells were used for each unique condition. Solution containing the test compound was added to the wells using a FLEXStation II (Molecular Devices, Sunnyvale, Calif.). Using the instrument in kinetic mode in which each well is read every 2 sec over a total collection time of 50 sec, fluorescence was measured using an excitation wavelength 494 nm and a detection wavelength of 516 nm. A normalized fluorescence value for each well was calculated using the following procedure. The difference in fluorescence at 35 sec (usually representing maximal response) and at 15 sec (a time point prior to addition of test compounds) is calculated, divided by the fluorescence at 15 sec, and the result multiplied by 100. The final value represents the percent increase in fluorescence relative to the fluorescence at 15 sec. Data was analyzed using standard procedures.

### Example 3

### cAMP Assay for Determining GABA_{B} Receptor Agonist Activity

The following procedure was used to determine the level of intracellular cAMP. Recombinant HEK cells expressing the GABA_{B} R1a2 receptor were used in the experiments. cAMP levels were measured using a cAMP XS⁺HitHunterm Chemiluminescence Assay Kit (90-0075-02, GE Healthcare Biosciences Corp.). Cells were seeded overnight at 5,000 cells per well, in black, clear bottom 96 well plates. The following morning, cells were washed twice with 100 μL PBS per well. Forskolin was weighed out and dissolved in DMSO to a final concentration 100 mM. 100 μM forskolin solutions were prepared in PBS with and without test compound at 1-times final concentration. 30 μL of the test solutions were added to the wells and incubated for 1 h at room temperature. After 1 h, the protocol described in the cAMP assay kit was followed, keeping the plate at room temperature and in the dark. Two hours after the final kit reagent was added, the plate bottom was covered with black tape, and the plate read using a 1450 MicroBeta Trilux microplate scintillation and luminescence counter (PerkinElmer, Waltham, Mass.). Each well was read for 6 seconds. The untransformed data was then analyzed.

### Example 4

### Preparation of a Sustained Release Oral Dosage Form of 1-{[(α-Isobutanovloxyethoxy)carbonyl]aminomethyl}-1-Cyclohexane Acetic Acid

Sustained release oral dosage forms containing the gabapentin prodrug, 1-{[(α-isobutanoyloxyethoxy)carbonyl]aminomethyl}-1-cyclohexane acetic acid (compound (7)), were prepared according the procedure disclosed in Cundy, U.S. Application Publication No. 2006/0141034, which is incorporated by reference herein in its entirety. Oral sustained release tablets containing compound (7) were made having the ingredients shown in Table 1:

TABLE 1
Ingredients of Oral Sustained Release Tablets

| | | Amount/ | Compo- | |
|---|---|---|---|---|
| | | Tablet | sition | Ingredient |
| Ingredient | Manufacturer | (mg/tablet) | (wt %) | Category |
| Compound (7) | XenoPort | 600.00 | 45.80 | Prodrug |
| | (Santa Clara, CA) | | | |
| Dibasic Calcium | Rhodia | 518.26 | 39.56 | Diluent |
| Phosphate, USP | (Chicago, IL) | | | |
| Glyceryl | Gattefosse | 60.05 | 4.58 | Lubricant/ |
| Behenate, NF | (Saint Pirest, | | | Release |
| | Cedex, France) | | | controlling |
| | | | | agent |
| Talc, USP | Barrett Minerals | 80.02 | 6.11 | Anti- |
| | (Mount Vernon, | | | adherent |
| | IN) | | | |
| Colloidal Silicon | Cabot | 5.43 | 0.41 | Glidant |
| Dioxide, NF | (Tuscola, IL) | | | |
| Sodium Lauryl | Fisher | 24.00 | 1.84 | Surfactant |
| Sulfate, NF | (Fairlawn, NJ) | | | |
| Magnesium | Mallinckrodt | 22.22 | 1.69 | Lubricant |
| Stearate, NF | (Phillipsburg, NJ) | | | |
| Total | | 1310.00 | 100 | |

The tablets were made according to the following steps. Compound (7), dibasic calcium phosphate, glyceryl behenate, talc, and colloidal silicon dioxide were weighed out, screened through a #20 mesh screen and mixed in a V-blender for 15 minutes. The slugging portion of the sodium lauryl sulfate was weighed and passed through a #30 mesh screen. The slugging portion of the magnesium stearate was weighed and passed through a #40 mesh screen. Screened sodium lauryl sulfate and magnesium stearate were added to the V-blender and blended for 5 min. The blend was discharged and compressed into slugs of approximately 400 mg weight on a tablet compression machine. The slugs were then passed through a Comil 194 Ultra mill (Quadro Engineering, Inc., Millburn, N.J.) to obtain the milled material for further compression. The tableting portion of the sodium lauryl sulfate was weighed and passed through a #30 mesh screen. The tableting portion of the magnesium stearate was weighed and passed through a #40 mesh screen. The milled material and the tableting portions of the sodium lauryl sulfate and magnesium stearate were added to the V-blender and blended for 3 min. The blended material was discharged and compressed to form tablets having a total weight of about 1310 mg and a compound (7) loading of about 600 mg (45.8 wt %). The tablets had a mean final hardness of 16.1 to 22.2 kp (158 to 218 Newtons). It will be appreciated that the sustained release oral dosage form may optionally be coated. For example, a tablet may be coated with Opadry II (39.3 mg/tablet).

### Example 5

### Pharmacokinetics of Orally Administered 1-{[(α-Isobutanoyloxyethoxy)carbonyl]aminomethyl}-1-Cyclohexane Acetic Acid

A randomized, crossover, fed/fasted single-dose study of the safety, tolerability, and pharmacokinetics of oral administration of 1-{[α-isobutanoyloxyethoxy)carbonyl]aminomethyl}-1-cyclohexane acetic acid (7) in healthy adult subjects was conducted. The oral sustained release dosage form of Example 4 was used in this study. The study was designed to evaluate the performance of this formulation in humans in comparison with the commercial gabapentin capsule formulation (Neurontin®, Pfizer). Twelve healthy adult volunteers (7 males and 5 females) participated in the study. Mean body weight was 75.6 kg. All subjects received two different treatments in a random order with a one-week washout between treatments. The two treatments were: a single oral dose of Example 4 tablets (2×600 mg) after an overnight fast; and a single oral dose of Example 4 tablets (2×600 mg) after a high fat breakfast.

Blood and plasma samples were collected from all subjects prior to dosing, and at 0.5, 1, 1.5, 2, 3, 4, 6, 8, 12, 18, 24, and 36 hours after dosing. Urine samples were collected from all subjects prior to dosing, and complete urine output was obtained at the 0-4 h, 4-8 h, 8-12 h, 12-18 h, 18-24 h, and 24-36 h intervals after dosing. Blood samples were quenched immediately with methanol and stored frozen at ≦70° C. Sample aliquots were prepared for analysis of gabapentin and compound (7) using sensitive and specific LC/MS/MS methods.

The mean±SD Cₘₐₓ for gabapentin in blood after oral dosing of the tablets (fasted) was 4.21±1.15 μg/mL. Following administration of the tablets after a high fat breakfast, the Cₘₐₓ of gabapentin in blood was further increased to 6.24±1.55 μg/mL. The mean±SD AUC for gabapentin in blood after oral dosing of the tablets (fasted) was 54.5±12.2 μg·h/mL. Following administration of the tablets after a high fat breakfast, the AUC of gabapentin in blood was further increased to 83.0±21.8 μg·h/mL. In the presence of food, exposure to gabapentin after oral administration of the tablets increased an additional 52% compared to that in fasted subjects.

The time to peak blood levels (Tₘₐₓ) of gabapentin was significantly delayed after oral administration of the tablets. In fasted subjects, oral administration of the tablets gave a gabapentin Tₘₐₓ of 5.08±1.62 h. This compares to a typical Tₘₐₓ of immediate release gabapentin of about 2-4 h. The gabapentin Tₘₐₓ after oral administration of the tablets was further delayed to 8.40±2.07 h in the presence of food. The apparent terminal elimination half-life for gabapentin in blood was similar for all treatments: 6.47±0.77 h for the tablets in fasted subjects, and 5.38±0.80 h for the tablets in fed subjects.

Following oral administration of the tablets, the percent of the gabapentin dose recovered in urine was 46.5±15.8% for fasted subjects and 73.7±7.2% for fed subjects.

Exposure to intact prodrug in blood after oral administration of the tablets was low. After oral dosing of the tablets in fasted subjects, concentrations of intact compound (7) in blood reached a maximum of 0.040 μg/mL, approximately 1.0% of the corresponding peak gabapentin concentration. Similarly, the AUC of compound (7) in blood of these subjects was 0.3% of the corresponding AUC of gabapentin in blood. After oral dosing of the tablets in fed subjects, concentrations of intact compound (7) in blood reached a maximum of 0.018 μg/mL, approximately 0.3% of the corresponding peak gabapentin concentration. Similarly, the AUC of compound (7) in blood of these subjects was less than 0.1% of the corresponding AUC of gabapentin in blood.

### Example 6

### Uptake of Gabapentin Following Administration of Gabapentin or Gabapentin Prodrugs Intracolonically in Rats

Sustained release oral dosage forms, which release drug slowly over periods of 6-24 hours, generally release a significant proportion of the dose within the colon. Thus drugs suitable for use in such dosage forms preferably exhibit good colonic absorption. This experiment was conducted to assess the suitability of gabapentin prodrugs for use in oral sustained release dosage forms.

### Step A: Administration Protocol

Rats were obtained commercially and were pre-cannulated in the both the ascending colon and the jugular vein. Animals were conscious at the time of the experiment. All animals were fasted overnight and until 4 hours post-dosing. Gabapentin or gabapentin prodrugs:
1-{[(α-benzoyloxybenzyloxy)carbonyl]aminomethyl}-1-cyclohexane acetic acid;
1-{[(α-benzoyloxyethoxy)carbonyl]aminomethyl}-1-cyclohexane acetic acid;
1-{[(α-benzoyloxybutoxy)carbonyl]aminomethyl}-1-cyclohexane acetic acid;
1-{[(α-propanoyloxyethoxy)carbonyl]aminomethyl}-1-cyclohexane acetic acid;
1-{[(α-isobutanoyloxyethoxy)carbonyl]aminomethyl}-1-cyclohexane acetic acid;
1-{[(α-acetoxyisobutoxy)carbonyl]aminomethyl}-1-cyclohexane acetic acid;
1-{[(α-acetoxyisopropoxy)carbonyl]aminomethyl}-1-cyclohexane acetic acid; and
1-{[((5-methyl-2-oxo-1,3-dioxol-4-en-4-yl)methoxy)carbonyl]aminomethyl}-1-cyclohexane acetic acid;

were administered as a solution (in water or PEG 400) directly into the colon via the cannula at a dose equivalent to 25 mg of gabapentin per kg. Blood samples (0.5 mL) were obtained from the jugular cannula at intervals over 8 hours and were quenched immediately by addition of acetonitrile/methanol to prevent further conversion of the prodrug. Blood samples were analyzed as described below.

### Step B: Sample Preparation for Colonic Absorbed Drug

In blank 1.5 mL Eppendorf tubes, 300 μL of 50/50 acetonitrile/methanol and 20 μL of p-chlorophenylalanine were added as an internal standard. Rat blood was collected at different time points and immediately 100 μL of blood was added into the Eppendorf tube and vortexed to mix 10 μL of a gabapentin standard solution (0.04, 0.2, 1, 5, 25, 100 μg/mL) was added to 90 μL of blank rat blood to make up a final calibration standard (0.004, 0.02, 0.1, 0.5, 2.5, 10 μg/mL). Then 300 μL of 50/50 acetonitrile/methanol was added into each tube followed by 20 μL of p-chlorophenylalanine. Samples were vortexed and centrifuged at 14,000 rpm for 10 min. Supernatant was taken for LC/MS/MS analysis.

### Step C: LC/MS/MS Analysis

An API 2000 LC/MS/MS spectrometer equipped with Shidmadzu 10ADVp binary pumps and a CTC HTS-PAL autosampler were used in the analysis. A Zorbax XDB C8 4.6×150 mm column was heated to 45° C. during the analysis. The mobile phase was 0.1% formic acid (A) and acetonitrile with 0.1% formic acid (B). The gradient condition was: 5% B for 1 min, then to 98% B in 3 min, then maintained at 98% B for 2.5 min. The mobile phase was returned to 5% B for 2 min. A TurbolonSpray source was used on the API 2000. The analysis was done in positive ion mode and an MRM transition of 172/137 was used in the analysis of gabapentin (MRM transitions:

m/z 426/198 for 1-{[(α-benzoyloxybenzyloxy)carbonyl]aminomethyl}-1-cyclohexane acetic acid;

m/z 364/198 for 1-{[(α-benzoyloxyethoxy)carbonyl]aminomethyl}-1-cyclohexane acetic acid;

m/z 392/198 for 1-{[(α-benzoyloxybutoxy)carbonyl]aminomethyl}-1-cyclohexane acetic acid;

m/z 316/198 for 1-{[(α-propanoyloxyethoxy)carbonyl]aminomethyl}-1-cyclohexane acetic acid;

m/z 330/198 for 1-{[(α-isobutanoyloxyethoxy)carbonyl]aminomethyl}-1-cyclohexane acetic acid;

m/z 330/198 for 1-{[(α-acetoxyisobutoxy)carbonyl]aminomethyl}-1-cyclohexane acetic acid;

m/z 316/198 for 1-{[(α-acetoxyisopropoxy)carbonyl]aminomethyl}-1-cyclohexane acetic acid; and

m/z 327.7/153.8 for 1-{[((5-methyl-2-oxo-1,3-dioxol-4-en-4-yl)methoxy)carbonyl]aminomethyl}-1-cyclohexane acetic acid;

were used. 20 μL of the samples were injected. The peaks were integrated using Analyst 1.1 quantitation software. Following colonic administration of each of these prodrugs, the maximum plasma concentrations of gabapentin (Cₘₐₓ), as well as the area under the gabapentin plasma concentration vs. time curves (AUC) were significantly greater (>2-fold) than that produced from colonic administration of gabapentin itself. For example, prodrug 1-{[(o-isobutanoyloxyethoxy)carbonyl]aminomethyl}-1-cyclohexane acetic acid provided both gabapentin Cₘₐₓ and AUC values greater than 10-fold higher than gabapentin itself. This data demonstrates that compounds of the invention may be formulated as compositions suitable for enhanced absorption and/or effective sustained release of GABA analogs to minimize dosing frequency due to rapid systemic clearance of these GABA analogs.

### Example 7

### Uptake of Pregabalin Following Administration of Pregabalin or Pregabalin Prodrugs Intracolonically in Rats

The protocol of Example 5 was repeated with pregabalin and the pregabalin prodrugs 3-{[(α-isobutanoyloxyethoxy)carbonyl]aminomethyl](3S)-5-methylhexanoic acid and 3-{[(α-isobutanoyloxyisobutoxy)carbonyl]aminomethyl](3S)-5-methyl-hexanoic acid. Following colonic administration of each of these prodrugs, the maximum plasma concentrations of pregabalin (Cₘₐₓ), as well as the area under the pregabalin plasma concentration vs. time curves (AUC) were significantly greater (>2-fold) than that produced from colonic administration of pregabalin itself.

### Example 8

### Uptake of R-Baclofen Following Administration of R-Baclofen or R-Baclofen Prodrugs Intracolonically in Rats

Sustained release oral dosage forms, which release drug slowly over periods of 6-24 hours, generally release a significant proportion of the dose within the colon. Thus, drugs suitable for use in such dosage forms preferably exhibit good colonic absorption. This experiment was conducted to assess the suitability of baclofen prodrugs for use in oral sustained release dosage forms.

### Step A: Administration Protocol

Rats were obtained commercially and were pre-cannulated in the both the ascending colon and the jugular vein. Animals were conscious at the time of the experiment. All animals were fasted overnight and until 4 hours post-dosing. R-Baclofen or baclofen prodrugs:

sodium 4-[(acetoxymethoxy)carbonylamino]-(3R)-(4-chlorophenyl)-butanoate;

sodium 4-[(benzoyloxymethoxy)carbonylamino]-(3R)-(4-chlorophenyl)-butanoate;

sodium 4-[(1-acetoxyisobutoxy)carbonylamino]-(3R)-(4-chlorophenyl)-butanoate;

sodium 4-[(1-isobutanoyloxyisobutoxy)carbonylamino]-(3R)-(4-chlorophenyl)-butanoate;

sodium 4-[(1-butanoyloxyisobutoxy)carbonylamino]-(3R)-(4-chlorophenyl)-butanoate;

sodium 4-[(1-butanoyloxyethoxy)carbonylamino]-(3R)-(4-chlorophenyl)-butanoate;

sodium 4-[(1-isobutanoyloxyethoxy)carbonylamino]-(3R)-(4-chlorophenyl)-butanoate;

sodium 4-[(1-benzoyloxyethoxy)carbonylamino]-(3R)-(4-chlorophenyl)-butanoate;

sodium 4-[(2,2-diethoxypropanoyloxymethoxy)carbonylamino]-(3R)-(4-chlorophenyl)-butanoate;

sodium 4-{[(1S)-isobutanoyloxyisobutoxy]carbonylamino}-(3R)-(4-chlorophenyl)-butanoate;

sodium 4-{[(1R)-isobutanoyloxyisobutoxy]carbonylamino}-(3R)-(4-chlorophenyl)-butanoate; and
(4-{[(1S)-isobutanoyloxyisobutoxy]carbonylamino}-(3R)-(4-chlorophenyl)-butanoic acid;

were administered as a solution (in water or PEG 400) directly into the colon via the cannula at a dose equivalent to 10 mg of baclofen equivalents per kg body weight. Blood samples (0.5 mL) were obtained from the jugular cannula at intervals over 8 hours and were quenched immediately by addition of methanol to prevent further conversion of the prodrug. Blood samples were analyzed as described below.

### Step B: Sample Preparation for Colonic Absorbed Drug

Rat blood was collected at different time points and 100 μL of blood was added into an Eppendorf tube containing 300 μL of methanol and vortexed to mix immediately. Twenty (20) μL of p-chlorophenylalanine was added as an internal standard. 300 μL of methanol was added into each tube followed by 20 μL of p-chlorophenylalanine. 90 μL of blank rat blood was added to each tube and mix. Then 10 μL of a baclofen standard solution (0.04, 0.2, 1, 5, 25, 100 μg/mL) was added to make up a final calibration standard (0.004, 0.02, 0.1, 0.5, 2.5, 10 μg/mL). Samples were vortexed and centrifuged at 14,000 rpm for 10 min. Supernatant was taken for LC/MS/MS analysis.

### Step C: LC/MS/MS Analysis

An API 2000 LC/MS/MS spectrometer equipped with Shidmadzu 10ADVp binary pumps and a CTC HTS-PAL autosampler were used in the analysis. A Phenomenex hydro-RP 4.6×50 mm column was used during the analysis. The mobile phase was water with 0.1% formic acid (A) and acetonitrile with 0.1% formic acid (B). The gradient condition was: 10% B for 0.5 min, then to 95% B in 2.5 min, then maintained at 95% B for 1.5 min. The mobile phase was returned to 10% B for 2 min. A TurboIonSpray source was used on the API 2000. The analysis was done in positive ion mode and an MRM transition of m/z 214/151 was used in the analysis of baclofen and MRM transitions:

m/z 330/240 for sodium 4-[(acetoxymethoxy)carbonylamino]-(3R)-(4-chlorophenyl)-butanoate;

m/z 392/240 for sodium 4-[(benzoyloxymethoxy)carbonylamino]-(3R)-(4-chlorophenyl)-butanoate;

m/z 372/240 for sodium 4-[(1-acetoxyisobutoxy)carbonylamino]-(3R)-(4-chlorophenyl)-butanoate;

m/z 400/240 for sodium 4-[(1-isobutanoyloxyisobutoxy)carbonylamino]-(3R)-(4-chlorophenyl)-butanoate;

m/z 400/240 for sodium 4-[(1-butanoyloxyisobutoxy)carbonylamino]-(3R)-(4-chlorophenyl)-butanoate;

m/z 372/240 for sodium 4-[(1-butanoyloxyethoxy)carbonylamino]-(3R)-(4-chlorophenyl)-butanoate;

m/z 372/240 for sodium 4-[(1-isobutanoyloxyethoxy)carbonylamino]-(3R)-(4-chlorophenyl)-butanoate,

m/z 406/240 for sodium 4-[(1-benzoyloxyethoxy)carbonylamino]-(3R)-(4-chlorophenyl)-butanoate;

m/z 454/61 for sodium 4-[(2,2-diethoxypropanoyloxymethoxy)carbonylamino]-(3R)-(4-chlorophenyl)-butanoate;

m/z 400/240 for sodium 4-{[(1S)-isobutanoyloxyisobutoxy]carbonylamino}-(3R)-(4-chlorophenyl)-butanoate;

m/z 400/240 for (sodium 4-{[(1R)-isobutanoyloxyisobutoxy]carbonylamino}-(3R)-(4-chlorophenyl)-butanoate; and

m/z 400/240 for 4-{[(1S)-isobutanoyloxyisobutoxy]carbonylamino}-(3R)-(4-chlorophenyl)-butanoic acid;

were used. Ten (10) μL of the samples were injected. The peaks were integrated using Analyst 1.2 quantitation software. Following colonic administration of prodrugs:

sodium 4-[(benzoyloxymethoxy)carbonylamino]-(3R)-(4-chlorophenyl)-butanoate;

sodium 4-[(1-acetoxyisobutoxy)carbonylamino]-(3R)-(4-chlorophenyl)-butanoate;

sodium 4-[(1-isobutanoyloxyisobutoxy)carbonylamino]-(3R)-(4-chlorophenyl)-butanoate;

sodium 4-[(1-butanoyloxyisobutoxy)carbonylamino]-(3R)-(4-chlorophenyl)-butanoate;

sodium 4-[(1-butanoyloxyethoxy)carbonylamino]-(3R)-(4-chlorophenyl)-butanoate;

sodium 4-[(1-isobutanoyloxyethoxy)carbonylamino]-(3R)-(4-chlorophenyl)-butanoate;

sodium 4-[(1-benzoyloxyethoxy)carbonylamino]-(3R)-(4-chlorophenyl)-butanoate;

sodium 4-[(2,2-diethoxypropanoyloxymethoxy)carbonylamino]-(3R)-(4-chlorophenyl)-butanoate;

sodium 4-{[(1S)-isobutanoyloxyisobutoxy]carbonylamino}-(3R)-(4-chlorophenyl)-butanoate;

sodium 4-{[(1R)-isobutanoyloxyisobutoxy]carbonylamino}-(3R)-(4-chlorophenyl)-butanoate; and
(4-{[(1S)-isobutanoyloxyisobutoxy]carbonylamino}-(3R)-(4-chlorophenyl)-butanoic acid;

the maximum plasma concentrations of R-baclofen (Cₘₐₓ), as well as the area under the baclofen plasma concentration vs. time curves (AUC) were significantly greater (>2-fold) than that produced from colonic administration of R-baclofen itself. This data demonstrates that these compounds may be formulated as compositions suitable for enhanced absorption and/or effective sustained release of baclofen analogs to minimize dosing frequency due to rapid systemic clearance of these baclofen analogs.

### Example 9

### Uptake of R-Baclofen Following Administration of R-Baclofen or R-Baclofen Prodrugs Intracolonically in Cynomolgus Monkeys

R-Baclofen hydrochloride salt and R-baclofen prodrugs (5 mg baclofen-eq/kg) were administered to groups of four male cynomolgus monkeys as either aqueous solutions or suspensions in 0.5% methyl cellulose/0.1% Tween-80 via bolus injection directly into the colon via an indwelling cannula. For colonic delivery a flexible French catheter was inserted into the rectum of each monkey and extended to the proximal colon (approx. 16 inches) using fluoroscopy. Monkeys were lightly sedated by administration of Telazol/ketamine during dosing. A washout period of at least 5 to 7 days was allowed between treatments. Following dosing, blood samples were obtained at intervals over 24 hours and were immediately quenched and processed for plasma at 4° C. All plasma samples were subsequently analyzed for R-baclofen and intact prodrug using the LC/MS/MS assay described above. Following colonic administration of prodrugs:

sodium 4-[(benzoyloxymethoxy)carbonylamino]-(3R)-(4-chlorophenyl)-butanoate;

(benzyl 4-[(1-acetoxyisobutoxy)carbonylamino]-(3R)-(4-chlorophenyl)-butanoate;

sodium 4-[(1-isobutanoyloxyisobutoxy)carbonylamino]-(3R)-(4-chlorophenyl)-butanoate;

sodium 4-[(1-benzoyloxyethoxy)carbonylamino]-(3R)-(4-chlorophenyl)-butanoate; and

sodium 4-[(2,2-diethoxypropanoyloxymethoxy)carbonylamino]-(3R)-(4-chlorophenyl)-butanoate;

the maximum plasma concentrations of baclofen (Cₘₐₓ), as well as the area under the baclofen plasma concentration vs. time curves (AUC) were significantly greater (>2-fold) than that produced from colonic administration of R-baclofen itself, while colonic administration of:

sodium 4-{[(1S)-isobutanoyloxyisobutoxy]carbonylamino}-(3R)-(4-chlorophenyl)-butanoate;

sodium 4-{[(1R)-isobutanoyloxyisobutoxy]carbonylamino}-(3R)-(4-chlorophenyl)-butanoate; and
(4-{[(1S)-isobutanoyloxyisobutoxy]carbonylamino}-(3R)-(4-chlorophenyl)-butanoic acid;

produced R-baclofen exposures that were greater than 10-fold that produced from colonic administration of R-baclofen itself. This data demonstrates that these compounds may be formulated as compositions suitable for enhanced absorption and/or effective sustained release of baclofen analogs to minimize dosing frequency due to rapid systemic clearance of these baclofen analogs.

### Example 10

### Uptake of R-Baclofen Following Oral Administration of R-Baclofen Prodrugs to Cynomolgus Monkeys

The R-baclofen prodrugs:

sodium 4-{[(1S)-isobutanoyloxyisobutoxy]carbonylamino}-(3R)-(4-chlorophenyl)-butanoate; and
4-{[(1S)-isobutanoyloxyisobutoxy]carbonylamino}-(3R)-(4-chlorophenyl)-butanoic acid;

(5 mg baclofen-eq/kg) were administered by oral gavage to groups of four male cynomolgus monkeys as either an aqueous solution or suspension in 0.5% methylcellulose/0.1% Tween-80 respectively. Following dosing, blood samples were obtained at intervals over 24 hours and were immediately quenched and processed for plasma at 4° C. All plasma samples were subsequently analyzed for R-baclofen and intact prodrug using the LC/MS/MS assay described above. The oral bioavailability of both prodrugs:
(sodium 4-{[(1S)-isobutanoyloxyisobutoxy]carbonylamino}-(3R)-(4-chlorophenyl)-butanoate; and
4-{[(1S)-isobutanoyloxyisobutoxy]carbonylamino}-(3R)-(4-chlorophenyl)-butanoic acid;

as R-baclofen was determined to be greater than 80%.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

What is claimed is:

1. A method of treating spasticity in a patient comprising administering to a patient in need of such treatment a colonically absorbable prodrug of a GABA analog having antispastic activity that is not directly mediated by the GABA_{B} receptor and an antispasticity agent, wherein the combined amounts are therapeutically effective.

2. The method of claim 1, wherein the colonically absorbable prodrug of a GABA analog is chosen from a colonically absorbable prodrug of gabapentin and a colonically absorbable prodrug of pregabalin.

3. The method of claim 2, wherein the colonically absorbable prodrug of gabapentin is chosen from a compound of Formula (I): 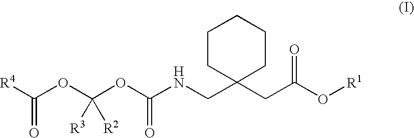
and the colonically absorbable prodrug of pregabalin is chosen from a compound of Formula (II): 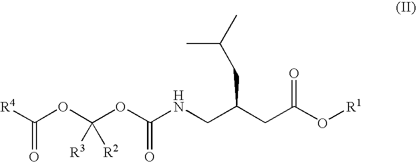
and a pharmaceutically acceptable salt of any of the foregoing, wherein:
R¹ is chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, heterocycloalkyl, substituted heterocycloalkyl, heteroaryl, substituted heteroaryl, heteroarylalkyl, and substituted heteroarylalkyl; or R² and R³ together with the carbon atom to which they are bonded form a ring chosen from a cycloalkyl, substituted cycloalkyl, heterocycloalkyl, and substituted heterocycloalkyl ring; and
R⁴ is chosen from acyl, substituted acyl, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl.

4. The method of claim 3, wherein R¹ is hydrogen.

5. The method of claim 3, wherein R² and R³ are independently chosen from hydrogen and C₁₋₆ alkyl.

6. The method of claim 3, wherein one of R² and R³ is C₁₋₆ alkyl and the other of R² and R³ is hydrogen.

7. The method of claim 3, wherein R⁴ is chosen from C₁₋₆ alkyl and substituted C₁₋₆ alkyl.

8. The method of claim 3, wherein each of R¹ and R² is hydrogen; R³ is C₁₋₆ alkyl; and R⁴ is chosen from C₁₋₆ alkyl and substituted C₁₋₆ alkyl.

9. The method of claim 3, wherein the compound of Formula (I) is 1-{[(α-isobutanoyloxyethoxy)carbonyl]aminomethyl}-1-cyclohexane acetic acid and the compound of Formula (II) is 3-{[(α-isobutanoyloxyethoxy)carbonyl]aminomethyl}(3S)-5-methyl hexanoic acid.

10. The method of claim 1, wherein the antispasticity agent is chosen from baclofen, R-baclofen, diazepam, tizanidine, clonidine, dantrolene, 4-aminopyridine, cyclobenzaprine, ketazolam, tiagabine, botulinum A toxin, and a prodrug of any of the foregoing.

11. The method of claim 1, wherein the antispasticity agent is a colonically absorbable prodrug of a GABA_{B} receptor agonist.

12. The method of claim 11, wherein the GABA_{B} receptor agonist is R-baclofen.

13. The method of claim 11, wherein the colonically absorbable prodrug of a GABA_{B} receptor agonist is chosen from a compound of Formula (IV): 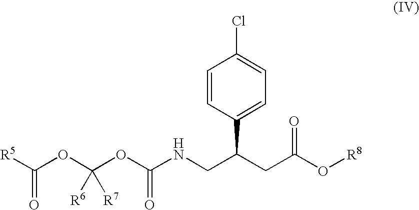
and a pharmaceutically acceptable salt thereof, wherein:
R⁵ is chosen from acyl, substituted acyl, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;
R⁶ and R⁷ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl; or R⁶ and R⁷ together with the carbon atom to which they are bonded form a ring chosen from a cycloalkyl, substituted cycloalkyl, heterocycloalkyl, and substituted heterocycloalkyl ring; and
R⁸ is chosen from hydrogen, alkyl, substituted alkyl, aryl, substituted aryl, arylalkyl, substituted arylalkyl, aryldialkylsilyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, and trialkylsilyl.

14. The method of claim 13, wherein R⁸ is hydrogen.

15. The method of claim 13, wherein R⁶ and R⁷ are independently chosen from hydrogen and C₁₋₆ alkyl.

16. The method of claim 13, wherein one of R⁶ and R⁷ is C₁₋₆ alkyl and the other of R⁶ and R⁷ is hydrogen.

17. The method of claim 13, wherein R⁵ is chosen from C₁₋₆ alkyl and substituted C₁₋₆ alkyl.

18. The method of claim 13, wherein R⁵ is C₁₋₆ alkyl; R⁶ is C₁₋₆ alkyl; R⁷ is hydrogen; and R⁸ is hydrogen.

19. The method of claim 13, wherein the compound of Formula (IV) is (3R)-4-{[(1S)-2-methyl-1-(2-methylpropanoyloxy)propoxy]carbonylamino}-3-(4-chlorophenyl)butanoic acid or a pharmaceutically acceptable salt thereof.

20. The method of claim 1, wherein the colonically absorbable prodrug of a GABA analog is administered orally.

21. The method of claim 20, comprising administering the colonically absorbable prodrug of a GABA analog in a sustained release oral dosage form.

22. The method of claim 1, wherein a therapeutically effective amount of the GABA analog and a therapeutically effective amount of the antispasticity agent are maintained in the plasma of the patient for a period of at least about 4 hours after administrating the colonically absorbable prodrug of a GABA analog and the antispasticity agent.

23. A method of treating spasticity in a patient comprising administering to a patient in need of such treatment a colonically absorbable prodrug of gabapentin of Formula (I): 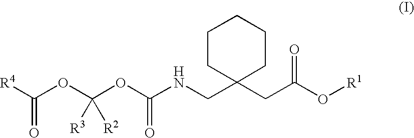
or a pharmaceutically acceptable salt thereof, wherein each of R¹ and R² is hydrogen; R³ is C₁₋₆ alkyl; and R⁴ is chosen from C₁₋₆ alkyl and substituted C₁₋₆ alkyl; and a colonically absorbable prodrug of a GABA_{B} agonist of Formula (IV): 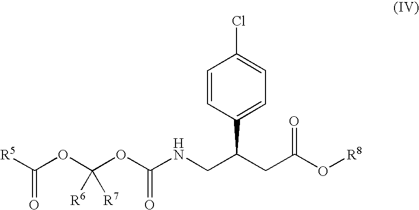
or a pharmaceutically acceptable salt thereof, wherein each of R⁷ and R⁸ is hydrogen; R⁶ is C₁₋₆ alkyl; and R⁵ is chosen from C₁₋₆ alkyl and substituted C₁₋₆ alkyl.

24. A method of treating spasticity in a patient comprising administering to a patient in need of such treatment a colonically absorbable prodrug of pregabalin of Formula (II): 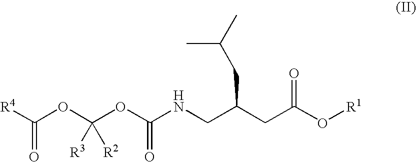
or a pharmaceutically acceptable salt thereof, wherein each of R¹ and R² is hydrogen; R³ is C₁₋₆ alkyl; and R⁴ is chosen from C₁₋₆ alkyl and substituted C₁₋₆ alkyl; and a colonically absorbable prodrug of a GABA_{B} agonist of Formula (IV): 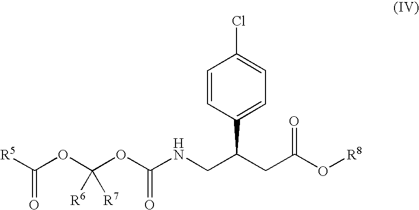
or a pharmaceutically acceptable salt thereof, wherein each of R⁷ and R⁸ is hydrogen; R⁶ is C₁₋₆ alkyl; and R⁵ is chosen from C₁₋₆ alkyl and substituted C₁₋₆ alkyl.
